(19) United States
(12) Patent Application Publication
(10) Pub. No.: US 2003/0203899 A1
(43) Pub. Date: Oct. 30, 2003

(54) Drugs for incontinence

(76) Inventors: Piero Del Soldato, Monza (IT); Francesca Benedini, Milano (IT)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/343,330

(22) PCT Filed: Jul. 27, 2001
(86) PCT No.: PCT/EP01/08734

(30) Foreign Application Priority Data
Aug. 8, 2000 (IT) MI2000 A 001848

(51) Int. Cl. A61K 31/54; A61K 31/537; A61K 31/495; A61K 31/445; A61K 31/426; A61K 31/421; A61K 31/4172

(52) U.S. Cl. 514/227.5; 514/252.12; 514/237.8; 514/331; 514/365; 514/374; 514/400

(57) ABSTRACT

Use in the incontinence of one or more of the following classes of drugs selected from the following: B) salified and non salified nitric oxide-donor drugs, of formula: A-X₁—N(O)_{z}, B′) nitrate salts of drugs used for the incontinence, and which do not contain in the molecule a nitric oxide donor group; C) organic or inorganic salts of compounds inhibiting phosphodiesterases.

The present invention relates to the use of classes of drugs, optionally mixtures thereof, for the urinary incontinence therapy.

More specifically, the invention relates to the use in the urinary incontinence therapy of one or more of the following compounds as defined hereunder, characterized in that they have a good efficacy in the urinary incontinence treatment combined with low side effects.

It is well known that the urinary incontinence can be considered a micturition control trouble consequent on a lesion or a dysfunction of the low urinary ducts. In particular the smooth musculature of the urinary bladder, called detrusor muscle, and the internal urethral sphincters (smooth musculature) and external (striated musculature) are involved. See for example Ferguson D. and Christopher N., Urinary Bladder Function and Drug Development, Trends in Pharmacological Sciences, 1996, 17, 161-165. In this publication it is mentioned that various kinds of incontinence exist characterized by different causes and symptoms. In particular it can be mentioned:

incontinence from efforts which consists in the loss of small amounts of urine as a consequence of an intrabdominal pressure increase, due to, for example, a cough or an effort. It is due to the change of the vesico-urethral angle and to the musculature relaxation of the urethral sphincters. It is frequent above all in multipara women;

incontinence from urgency which consists in the inability to control the bladder and it appears with a sudden and impelling stimolus to urinate. It is due to intermittent contractions of the bladder musculature without evident causes (detrusor instability) or consequent on interstitial cystitis or other inflammatory phenomena which cause bladder hyperexcitability. It seems that in all these cases alterations of the bladder innervation are present;

incontinence from bladder overrelaxation which appears in the cases of chronic urinary retention due to obstructive causes. The bladder never empties itself completely with consequent continuous loss of small amounts of urine;

total incontinence which consists in the complete lack of control on the bladder due to inability to control the sphincters. It is a consequence of serious neurological damages.

In the prior art the available therapies are based on three different approaches—see for example the above article and Anderson K. E., Pharmacology of Lower Urinary Tract Smooth Muscles and Penile Erectile Tissues, Pharmacological Reviews, 1993, 45, 253-308:

reduction of the detrusor activity,

modification of the sensory nervous transmission,

modification of the urethral resistances.

According to the first approach, the detrusor contraction is stimulated by the parasympathetic system and acetylcholine is the main mediator. Therefore to reduce the bladder hyperactivity anticholinergic drugs are used which are effective but of limited use owing to the anticholinergic activity at systemic level. Indeed they cause side effects such as for example fauces dryness, constipation and tachycardia. If one considers that the bladder irritability is often associated to obstructive bladder pathologies, the administration of anticholinergic drugs can potentially cause crises of acute urinary retention.

For example anticholinergic drugs such as oxybutynin or tolterodine are quite effective. Their use is however limited by the side effects typical of anticholinergic agents (fauces dryness, dimmed sight, etc.) Occasionally patients under treatment with said products can also have cardiac rhythm troubles. In patients affected by glaucoma, a worsening of the pathology can happen, furthermore in old patients with prostatic hypertrophy a worsening of the urinary retention can take place.

Another pharmacological approach for reducing the detrusor activity considers the use of drugs which facilitate the opening of the channels of potassium, of calcium antagonists and of relaxing drugs of the smooth musculature. Also in this case there are side effects, such as for example the arising of a marked hypotensive action due to the aspecific effect of vasodilation induced by these drugs.

The administration of S-agonist drugs induces an increase of the bladder capacity, but their use is limited by the serious side effects affecting the cardiovascular system.

A further pharmacological approach for reducing the bladder hyperactivity is the use of antidepressant drugs, but also with these therapeutic aids there are serious side effects affecting the cardiovascular system (orthostatic hypotension, arrhythmia).

Another pharmacological method for reducing the detrusor activity consists in the use of the prostglandin synthesis inhibitors which have been experimented in some cases of detrusor hyperactivity and enuresis with promising results. Also in this case the side effects which have been noticed have been significant. The use of these drugs is based on the fact that several prostglandines are synthesized at bladder level as a consequence of nervous stimulation and some of them would have the function of mediators of the detrusor muscle contractions. Some prostglandines would be furthermore involved in phenomena of incontinence from urgency and bladder hyperactivity noticed during some inflammatory pathologies of the urinary tract.

The non steroidal antiinflammatory drugs are potentially useful for reducing the limit of excitability of the urinary bladder, and are therefore effective in the cases of detrusor instability. Unfortunately they show the drawback that at active doses they are poorly tolerated especially at the gastrointestinal apparatus level.

The NO enzyme synthetase inhibitors could prevent the bladder hyperexcitability and hyperalgesia consequent on inflammatory phenomena such as interstitial cistitis; see Rice A. S. C., Topical Spinal Administration of a Nitric oxide Synthase Inibitor Prevents the Hyper-Reflexia Associated with a Pat Model of Persistent Visceral Pain, Neuroscience Letters, 1995, 187, 111-114. However, at present, therapeutically usable drugs of this kind do not exist because of the corresponding aspecificity of their pharmacological profile.

The second approach which consists in the modification of the sensory nervous transmission (in the cases when the urinary incontinence derives from lesions of the nervous system) implies the use of active drugs on the neurotransmission, for example of garia-aminobutyric acid (GABA), or peptides, or purines, which are important neurotransmitters at the urinary ducts level.

Also clinical studies which use capsaicin by intravescical instillation with sometimes positive results are known. However this treatment has limited clinical applications due to its transitory effect and besides obtainable only by local administration.

The third approach is based on the fact that at the urethra level the musculature tone is mediated by different neurotransmission systems, for example the adrenergic one by stimulation of the α receptors. To modify the urethral resistances α-agonist drugs are used with sometimes satisfactory results; they increase the pressure bearable by the urethra. However the use of these compounds is contraindicated in the case of obstructive pathologies of the bladder, in which even alpha-antagonist drugs are used. In these cases it is indeed frequent that an hyperactivity of the sphincters takes place, which hinders the regular bladder emptying, causing incontinence from urgency. Also in this case, as well as in the first above described approach, serious side effects of hypotensive type bound to the α-antagonist activity affecting the cardiocirculatory apparatus level are to be pointed out.

Up to now the commercially available drugs solve the problem only in a limited number of cases but generally inducing side effects also of a certain seriousness.

Good results have been obtained with products described in patent application WO 98/09948 in the name of the Applicant wherein nitroxyderivatives of particular classes of non steroidal antinflammatory drugs are used. These products are very good drugs for the incontinence treatment with low side effects, however they have the drawback to have to be mainly administered by os. When a parenteral administration is necessary (cases of bad absorption, seriously ill hospitalized patients where the administration by os cannot be carried out), it has been found that the products mentioned in said application are not administrable by parenteral route.

The Applicant has unexpectedly and surprisingly found compounds effective in the incontinence treatment and giving lower side effects, and are administrable also parenterally, therefore overcoming the drawbacks of the prior art.

An object of the present invention is the use in the incontinence of one or more of the following classes of drugs selected from the following:

A) nitric oxide donor drugs, optionally salified, of formula:

A-X₁—N(O)_{z}

wherein A, X₁, Z have the meaning defined below;

B′) nitrate salts of drugs used for the incontinence and which do not contain in the molecule a nitric oxide donor group;

C) organic or inorganic salts of compounds inhibiting phosphodiesterases;

in the compounds of general formula:

A-X₁—N(O)_{z}

z is an integer and is 1 or 2, preferably 2;

A=R(COXᵤ)ₜ and wherein t is an integer 0 or 1; u is 0 or 1;

X=O, NH, NR_{1c} wherein R_{1c} is a linear or branched C₁-C₁₀ alkyl;

X₁ is the following bivalent linking group:

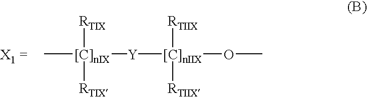

wherein:

nIX is an integer in the range 0-3, preferably 1;

nIIX is an integer in the range 1-3, preferably 1;

R_{TIX}, R_{TIX′}, R_{TIIX}, R_{TIIX′}, equal to or different from each other are H or linear or branched C₁-C₄ alkyl; preferably R_{TIX}, R_{TIX′}, R_{TIIX}, R_{TIIX′} are H;

Y is a heterocyclic ring containing one or two nitrogen atoms, optionally one oxygen or sulphur atom, said saturated, unsaturated or aromatic ring, having 5 or 6 atoms;

R is selected from the following groups:

Group I) wherein t 1 and u 1

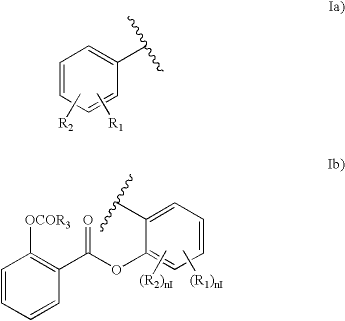

wherein:

R₁ is the OCOR₃ group; wherein R₃ is methyl, ethyl or linear or branched C₃-C₅ alkyl, or the residue of a heterocycle with only one ring having 5 or 6 atoms which can be aromatic, partially or totally hydrogenated, containing one or more heteroatoms independently selected from O, N and S;

R₂ is hydrogen, hydroxy, halogen, linear or branched when possible C₁-C₄ alkyl; a linear or branched when possible C₁-C₄ alkoxyl; a linear or branched when possible C₁-C₄ perfluoroalkyl, for example trifluoromethyl; nitro, amino, mono- or di-(C₁₋₄) alkylamino;

nI is an integer 0 or 1;

preferably in the compounds of formula Ia) X is equal to O or NH, R₁ is acetoxy, preferably in ortho position with respect to —CO—, R₂ is hydrogen; preferably X₁ is the linking group (B) wherein R_{TIX}=R_{TIX′}=R_{TIIX}=R_{TIIX′}=H, n_{IX}=n_{IIX}=1;

Preferably in the compounds of formula Ib) R₃=CH₃, nI=0, X is equal to O, X₁ is as above defined for Ia); in this case Ib) is the residue of the acetylsalicylsalicylic acid;

Group II, wherein t=1, u=1

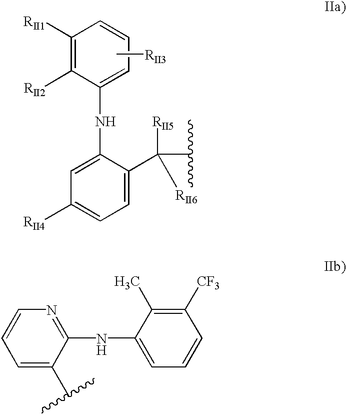

wherein:

R_{II5} is H, linear or branched when possible C₁-C₃ alkyl;

R_{II6} has the same meaning as R_{II5}, or when R_{II5} is H it can be benzyl;

R_{II1}, R_{II2} and R_{II3} can independently be hydrogen, linear or branched when possible C₁-C₆ alkyl, or linear or branched when possible C₁-C₆ alkoxy, or Cl, F, Br;

R_{II4} is R_{II1} or bromine;

the compounds wherein R_{II1}, R_{II4} are hydrogen and R_{II2} and R_{II3} are chlorine in ortho position with respect to NH are preferred; R_{II5} and R_{II6} are H, X is equal to O, and X₁ is as above defined for the compounds of formula Ia);

IIb) is the residue of the 2-[(2-methyl-3-(trifluoromethyl) phenyl]amino]-3-pyridincarboxylic] acid and when the —COOH group is present the compound is known as flunixin;

Group III) wherein t 1, u=1 and R is

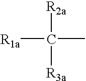

wherein:

R₂ₐ and R₃ₐ are H, linear or branched when possible, substituted or not, C₁-C₁₂ alkyl or allyl, with the proviso that if one of the two is allyl, the other is H; preferably R₂ₐ is H, C₁-C₄ alkyl, R₃ₐ is H;

R₁ₐ is selected from

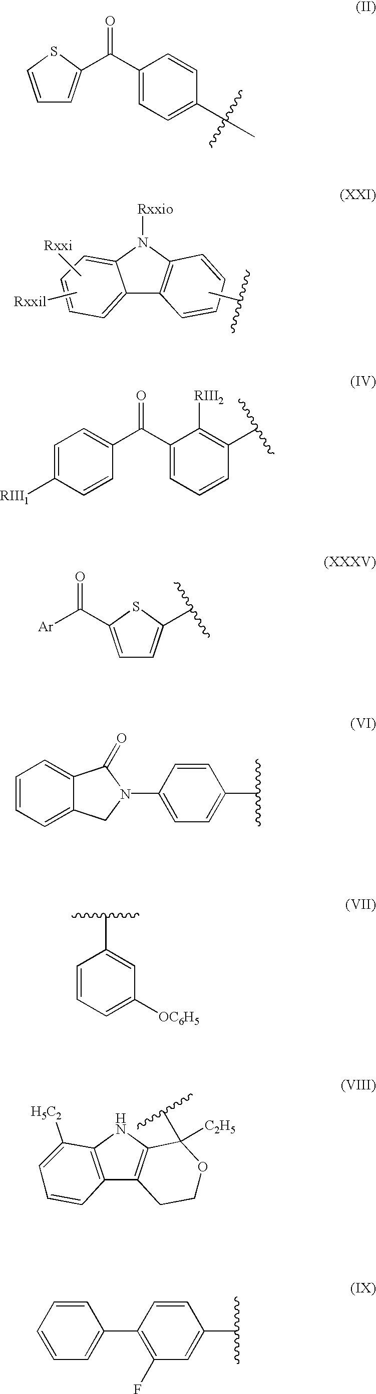

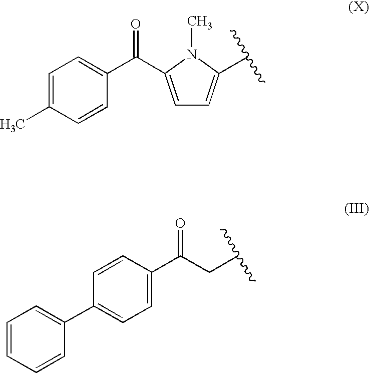

IIID) R₁ₐ corresponds to the following formulas:

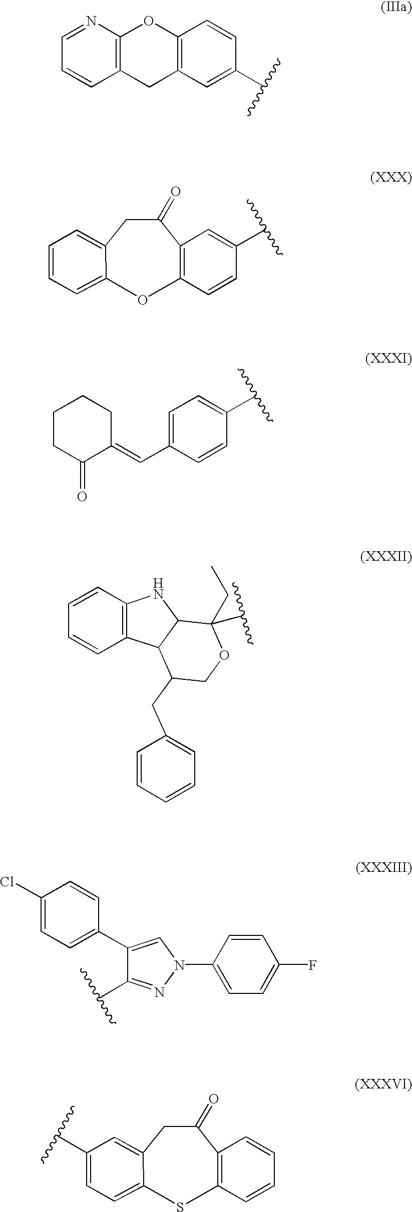

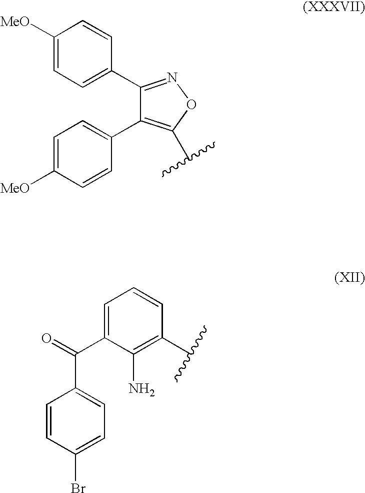

wherein the meanings are the following:

when R₁ₐ is as defined in formula (IV), Ketoprofen residue:

R_{III1} is H, SR_{III3} wherein R_{III3} contains from 1 to 4 carbon atoms, linear or branched when possible;

R_{III2} is H, hydroxy;

the compounds wherein R_{III1} and R_{III2} are H, R₃ₐ is H, and R₂ₐ is methyl, X=O, are preferred;

when R₁ₐ is as adefined in formula (XXI), carprofen residue:

Rₓₓᵢₒ is H, linear or branched when possible alkyl from 1 to 6 carbon atoms, C₁-C₆ alkoxycarbonyl linked to a C₁-C₆ alkyl, C₁-C₆ carboxyalkyl, C₁-C₆ alkanoyl, optionally substituted with halogens, benzyl or halobenzyl, benzoyl or halobenzoyl;

Rₓₓᵢ is H, halogen, hydroxy, CN, C₁-C₆ alkyl optionally containing OH groups, C₁-C₆ alkoxy, acetyl, benzyloxy, SRₓₓᵢ₂ wherein Rₓₓᵢ₂ is C₁-C₆ alkyl; C₁-C₃ perfluoroalkyl; C₁-C₆ carboxyalkyl optionally containing OH groups, NO₂, amino; sulphamoyl, di-alkyl sulphamoyl with C₁-C₆ alkyl, or difluoroalkylsulphonyl with C₁-C₃ alkyl;

Rₓₓᵢ₁ is halogen, CN, C₁-C₆ alkyl containing one or more OH groups, C₁-C₆ alkoxy, acetyl, acetamido, benzyloxy, SR_{III3} being R_{III3} as above defined, C₁-C₃ perfluoroalkyl, hydroxy, C₁-C₆ carboxyalkyl, NO₂, amino, mono- or di-alkyl-amino C₁-C₆; sulphamoyl, di-alkyl sulphamoyl C₁-C₆, or di-fluoroalkylsulphamoyl as above defined; or Rₓₓᵢ together with Rₓₓᵢ₁ is a C₁-C₆ alkylen dioxy;

the compounds are preferred wherein Rₓₓᵢₒ is H, the linking group is in position 2, Rₓₓᵢ is H, Rₓₓᵢ₁ is chlorine and is in para position with respect to nitrogen;

R₃ₐ is H, R₂ₐ is methyl and X is O;

when R₁ₐ is as defined in formula (XXXV), tiaprofenic acid residue:

Ar is phenyl, hydroxyphenyl optionally mono- or polysubstituted with halogen, alkanoyl and alkoxy C₁-C₆, C₁-C₆, preferably C₁C₃, trialkyl, cyclopentyl, cyclohexyl, cycloheptyl, heteroaryl, preferably thienyl, furyl optionally containing OH, pyridyl;

the preferred compounds of (XXXV) are those wherein Ar is phenyl, R₃ₐ is H, R₂ₐ is methyl and X is O;

when R₁ₐ is as defined in formula (II), suprofen residue,

of which the preferred one has been indicated, wherein R₃ₐ is H, R₂ₐ is methyl and X=O, as described and obtained in U.S. Pat. No. 4,035,376 herein incorporated by reference;

when R₁ₐ is as defined in formula (VI), R is the residue of indoprofen when R₂ₐ=H and R₃ₐ=CH₃; of indobufen when R₂ₐ is equal to H and R₃ₐ=C₂H₅; X=O, as described and obtained according to U.S. Pat. No. 3,997,669 herein incorported by reference;

when R₁ₐ is as defined in formula (VIII), R is the etodolac residue when R₂ₐ=R₃ₐ=H and X=O. as described and obtained according to U.S. Pat. No. 3,843,681 herein incorporated by reference;.

when R₁ₐ is as defined in formula (VII), R is the fenoprofen residue when R₃ₐ=H, R₂ₐ=CH₃ and X=O, as described and obtained according to U.S. Pat. No. 3,600,437 herein incorporated by reference;

when R₁ₐ is as defined in formula (III), R is the fenbufen residue when R₂ₐ=R₃ₐ=H and X=O, as described and obtained according to U.S. Pat. No. 3,784,701 herein incorporated by reference;

when R₁ₐ is as defined in formula (IX), R is the flurbiprofen residue when R₃ₐ=H, R₂ₐ=CH₃, X=O;

when R₁ₐ is as defined in formula (X) R is the tolmetin residue when R₂ₐ=R₃ₐ=H, X=O, as described and obtained according to FR 1,574,570 herein incorporated by reference;

In group IIID) R₁ₐ corresponds to the following formulas:

IIIa), when R₂ₐ=H and R₃ₐ=CH₃ the pranoprofen residue is obtained: α-methyl-5H-[1]benzopyran-[2,3-b]pyridin-7-acetic acid; the preferred compound has P, ═H, R₃ₐ=CH₃, u=1 and X=O:

(XXX), when R₂ₐ=H and R₃ₐ=CH₃, the bermoprofen residue is obtained: dibenz[b,f]oxepin-2-acetic acid; the preferred compound has. R₂ₐ=H, R₃ₐ=CH₃, u=1 and X=O.

(XXXI), when R₂ₐ=H and R₃ₐ=CH₃, R is the radical of the compound CS-670: 2-[4-(2-oxo-1-cyclohexyliden methyl) phenyl]propionic acid; the preferred compound has R₂ₐ=H, R₃ₐ=CH₃, u=1 and X=O;

(XXXII), when R₂ₐ═R₃ₐ=H, the Pemedolac residue is obtained; the preferred compound has R₂ₐ=R₃ₐ=H, u=1 and X=O;

(XXXIII), when R₂ₐ=R₃ₐ=H, the pirazolac residue is obtained: 4-(4-chlorophenyl)-1-(4-fluorophenyl)-3-pyrazolic acid derivatives;

The preferred compounds have R₂ₐ=R₃ₐ=H, u=1 and X=O;

(XXXVI), when R₂ₐ=H, R₃ₐ=CH₃, the zaltoprofen residue is obtained; when the residue is saturated with a hydroxyl or amino group, or with the carboxylic function the compounds are known as dibenzothiepine derivatives; the preferred compounds have R₂ₐ=H, R₃ₐCH₃, u=1 and X=O;

(XXXVII), when R₂ₐ=R₃ₐ=H the mofezolac residue is obtained: 3,4-di(p-methoxyphenyl)isoxazol-5-acetic acid when the residue is CH₂—COOH; the preferred compounds have R₂ₐ=R₃ₐ=H, t=1 and X=O;

(XII), when R₂ₐ=R₃ₐ=H the bromfenac residue is obtained: 2-amino-3-(4-bromobenzoyl)benzeneacetic acid;

the preferred compounds have u=1, t=1, X=O, R₂ₐ=R₃ₐ=H; or t=0;

in group IV) wherein t=1, u=1, R is

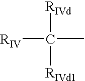

wherein:

R_{Ivd} and R_{Ivd1} are at least one H and the other a linear or branched when possible C₁-C₆, preferably C₁ and C₂ alkyl, or difluoroalkyl with the alkyl from 1 to 6 carbon atoms, C₁ is preferred, or R_{Ivd} and R_{Ivd1} form together a methylene group;

R_{IV} has the following meaning:

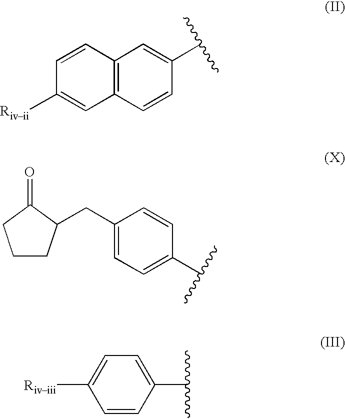

wherein the compounds of group IV) have the following meanings:

in formula (II)

R_{iV-ii} is C₁-C₆ alkyl, C₃-C₇ cycloalkyl, C₁-C₇ alkoxymethyl, C₁-C₃ trifluoroalkyl, vinyl, ethynyl, halogen, C₁-C₆ alkoxy, difluoroalkoxy, with C₁-C₇ alkyl, C₁-C₇ alkoxymethyloxy, alkylthiomethyloxy with C₁-C₇ alkyl, alkyl methylthio with C₁-C₇ alkyl, cyan, difluoromethylthio, phenyl- or phenylalkyl substituted with C₁-C₈ alkyl; preferably R_{iV-ii} is CH₃O—, R_{IVd} is H and R_{IVd1} is CH₃, and it is known as naproxen residue;

X=O and X₁ is as above defined for Ia);

in formula (X), of which the loxoprofen residue, described in U.S. Pat. No. 4,161,538 herein incorporated by reference, has been indicated, the compounds wherein R_{IVd} is H and R_{IVd1} is CH₃, X=O and X₁ is as above defined for Ia) are preferred;

in formula (III):

R_{iV-iii} is a C₂-C₅ alkyl, optionally branched when possible, C₂ and C₃ alkyloxy, allyloxy, phenoxy, phenylthio, cycloalkyl from 5 to 7 carbon atoms, optionally substituted in position 1 by a C₁-C₂ alkyl; it is preferred the compound wherein R_{iV-iii} is

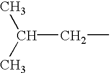

and R_{IVd}=H, R_{IVd1} is CH₃, compound known as ibuprofen residue; X=O and X₁ is as above defined for Ia)

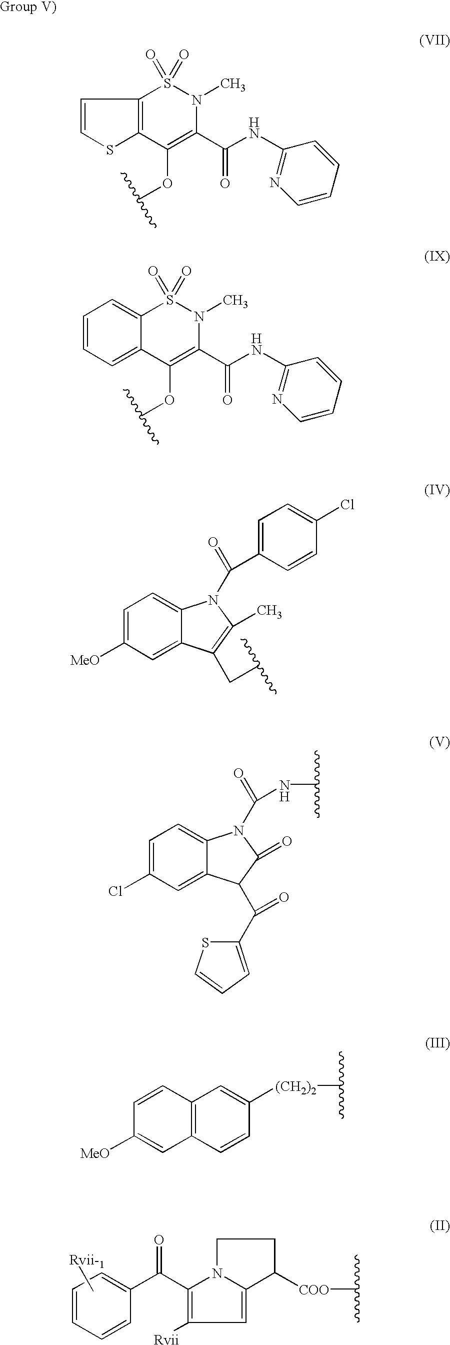

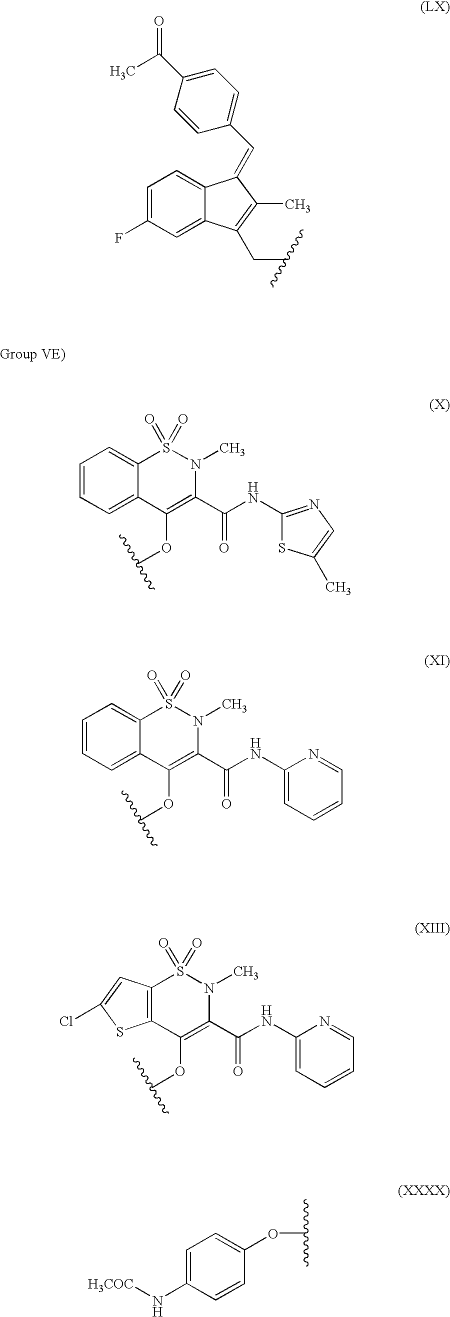

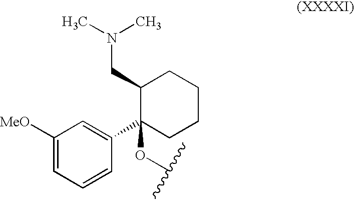

in group V), the compounds have the following meanings:

when R is formula (II),

R_{Vii} is H or a linear or branched when possible C₁-C₄ alkyl;

R_{Vii-1} is R_{Vii}, or a linear or branched when possible C₁-C₄ alkoxy; Cl, F, Br; the position of R_{Vii-1} being ortho, or metha, or para;

the residue of the known Ketorolac is preferred, wherein R_{Vii} and R_{Vii-1} are H, and A=R (A being the group of the formula A-X₁—NO₂) and t=0;

when R is formula (V),

of which the residue of the known tenidap has been indicated, as described and obtained in U.S. Pat. No. 4,556,672 herein incorporated by reference;

in these compounds of formula (V) A=R and t=0,

when R is formula (VII),

of which the residue of the known tenoxicam has been indicated, A is RCO, t=1 u=0 or A is R and t=0, as described and obtained in DE 2,537,070 herein incorporated by reference;

when R is formula (IX),

wherein A=R and t=0, or A=RCO with t=1 and u=0, the residue of the known piroxicam has been indicated, as described and obtained in U.S. Pat. No. 3,591,584 herein incorporated by reference;

when R is formula (III)

wherein A=RCOO, t=1 and u=0 or 1; or t=0 and A=R, of which the residue of the known nabumetone has been indicated, as described and obtained in U.S. Pat. No. 4,061,779 herein incorporated by reference;

when R is formula (IV)

wherein A=RCOO, t=1 and u=1, of which the indomethacin residue has been indicated, as described and obtained in U.S. Pat. No. 3,161,654, herein incorporated by reference;

when R=formula (LX) and in (COXᵤ)ₜ u=t=1 and X is oxygen, the precursor compound is known as sulindac;

when R is formula (X), the X residue is known as meloxicam; the preferred compounds are those wherein A=RCO, t=1 and u=0;

when R is formula (XI) the residue is known as ampiroxicam when the termination is —CH(CH₃)OCOC₂H₅; the preferred compounds have A=RCO, t=1 and u=0;

when R is formula (XIII) and the valence is saturated with H, the residue derives from lornoxicam; the preferred compounds have A=RCO, t=1 and u=0;

when R is formula (XXXX) and the valence is saturated with H the compound known as paracetamol is obtained, as described and obtained in U.S. Pat. No. 2,998,450 herein incorporated by reference;

when R is formula (XXXXI) and the valence is saturated with H, the compound known as Tramadol is obtained, as described and obtained in U.S. Pat. No. 3,652,589;

the preferred compounds according to the present invention obtainable with the radicals corresponding to the formulas (XXXX) and (XXXXI) have A=RCO, t=1 and u=0.

Preferably Y is selected from the following:

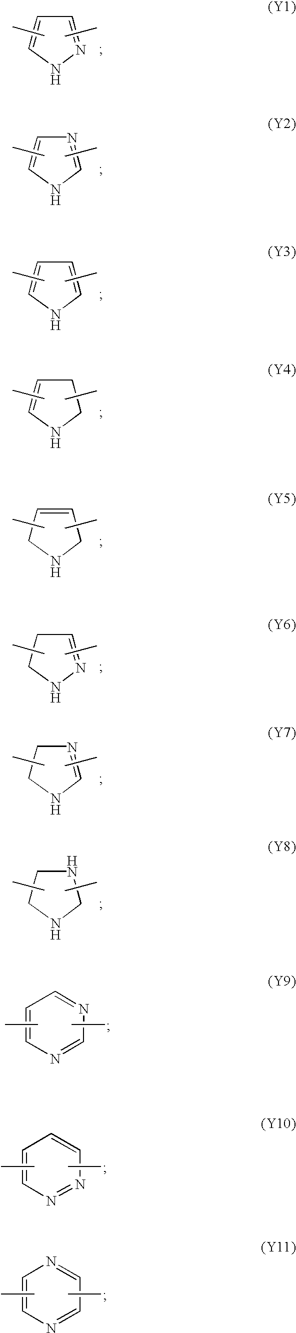

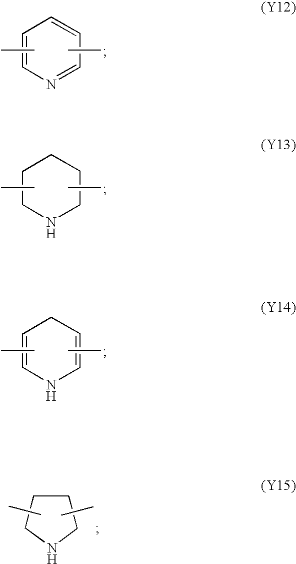

Preferably Y is an aromatic ring having 6 atoms, containing one nitrogen atom, said aromatic ring having the two free valences in position 2 and 6.

The preferred of Y is Y12 (pyridyl) substituted in position 2 and 6. The bonds can be also in a non symmetric position, for example Y12 (pyridyl) can be substituted also in position 2 and 3; Y1 (pyrazol) can be 3,5-disubstituted.

The X₁ precursors as defined by formula (B), wherein the free valence of the oxygen is saturated with H and the free valence of the end carbon is saturated with either a carboxylic or hydroxyl group, are commercially available compounds or they can be obtained by known methods of the prior art.

The compounds containing R of group I of the type Ia) are described in patent application WO 92/01668 wherein also the preparation methods are mentioned. This patent is herein incorporated by reference. The compounds of type Ib) are for example prepared by using the method indicated in The Merck Index, XI ed., 1989, pag. 16, No. 95 for the acetylsalicylsalicylic acid residue. The modifications of the compounds of formula Ib) can be obtained by using the processes mentioned in patent application WO 92/01668.

The compounds wherein R is of group II) are described in patent application WO 94/04484 and U.S. Pat. No. 3,558,690 wherein also the preparation methods are indicated. These patents are herein incorporated by reference.

The starting compound of IIb), when the valence is saturated with —COOH (flunixin), is obtained according to U.S. Pat. Nos. 3,337,570 and 3,689,653, both herein incorporated by reference. The compounds containing the substituents mentioned in the previous patents are equivalent to flunixin.

The compounds wherein R is of group III) are described and obtained by the processes mentioned in the following patents:

patent application PCT/EP/93 03193; for the compounds of formula (IV) see also U.S. Pat. No. 3,641,127; for the compounds of formula (XXI) see also U.S. Pat. No. 3,896,145; for the compounds of formula (IX) flurbiprofen residue see also U.S. Pat. No. 3,755,427; for the compounds of formula (II) see also U.S. Pat. No. 4,035,376; for the compounds of formula (VI) see also U.S. Pat. No. 3,997,669; for the compounds of formula (VIII) see also U.S. Pat. No. 3,843,681; for the compound of formula (VII) see also U.S. Pat. No. 3,600,437; for the compounds of formula (III) see also U.S. Pat. No. 3,784,701. All these mentioned patents are herein incorporated by reference.

The procedures for the preparation of the compounds of class IIID) are the following:

The residue IIIa) is obtained by preparing the acid compound according to U.S. Pat. No. 3,931,205, the valence is saturated with —CH(CH₃)—COOH. The compounds containing the substituents mentioned in the previous patent are equivalent to pranoprofen. The residue (XXX) is prepared through the compound with the group —CH(CH₃)—COOH (bermoprofen) according to U.S. Pat. No. 4,238,620 herein incorporated by reference. Other equivalent products are described in the above mentioned patent The residue (XXXI) is prepared by starting from the corresponding acid —CH(CH₃)—COOH according to U.S. Pat. No. 4,254,274. Equivalent compounds are described in the same patent.

The residue (XXXII) is prepared according to EP 238,226 herein incorporated by reference, when the valence is saturated with —CH₂—COOH. Equivalent products are reported in said patents as 1,3,4,9 tetrahydropyran [3,4-b] indol-1-acetic substituted acids.

The residue (XXXIII) is prepared from pirazolac and the valence is saturated with —CH₂—COOH, as indicated in EP 54,812 herein incorporated by reference. Equivalent products are described in said patent.

The residue (XXXVI) is prepared according to UK 2,035,311 herein incorporated by reference, by starting from zaltoprofen and having the —CH(CH₃)—COOH termination. Equivalent products are described in said patent.

The process for preparing the residue (XXXVII) is obtained starting from mofezolac and it is prepared according to EP 26,928. Equivalent products are reported in the same patent.

The compounds wherein R is of group IV) are described in GB patent application 2,283,238, wherein also the preparation methods are indicated; this patent is herein incorporated by reference.

In group IV) the compounds can also be obtained: for the compounds of formula (II) using U.S. Pat. No. 3,904,682; the compounds of formula (X) according to U.S. Pat. No. 4,161,538; the compounds of formula (III) according to U.S. Pat. No. 3,228,831. The herein mentioned patents are incorporated in the present application by reference.

In group V) the compounds can also be obtained: for the compounds of formula (II) using U.S. Pat. No. 4,089,969 herein incorporated by reference; the compounds of formula (V) can be obtained according to U.S. Pat. No. 4,556,672 herein incorporated by reference.

The residue (x) is prepared according to the German patent 2,756,113. Equivalent products are described in said patent.

The residue (XI) is prepared according to EP 147,177, herein incorporated by reference, starting from ampiroxicam having the termination —CH(CH₃)OCOOC₂H₅. Equivalent products are described in said patent.

The residue (XII) is prepared according to J. Med. Chem., vol. 27 No. 11, November 1984, Walsh et Al. “Antiinflammatory Agents. 3. Synthesis and Pharmacological Evaluation of 2-amino-3-benzoylphenylacetic Acid and Analogues”, herein incorporated by reference. Equivalent products are described in said publication.

The residue (XIII) is prepared starting from lornoxicam, wherein the valence is saturated with H. It is prepared according to GB 2,003,877. Equivalent products are described in said patent.

The residue (LX) in group V is prepared from Sulindac, obtained according to U.S. Pat. No. 3,654,349.

In general the connection between A and X₁ is, as seen, of ester or amidic type (NH or NR_{1c}, as defined in X) when R is of groups I, II, III, IV and V. For the formation of such connection all the synthesis routes well known for the formation of such bonds are usable.

The preparation of the compounds of formula A-X₁—N(O), with the linking group X₁ of formula (B) is described in published PCT application WO 00/51988 in the name of the Applicant, herein incorporated by reference.

The compounds of group A), as said, are effective in the incontinence treatment, they give lower side effects and are also parenterally administrable, therefore overcoming the drawbacks of the prior art mentioned in patent application WO 98/09948.

The drugs of the nitrate salts compounds B′) are selected from B′1) anticholinergic drugs, B′2) calcium-antagonist drugs, B′3) drugs which facilitate the opening of the potassium channels, B′4) alpha-adrenergic agonistic drugs, B′5) alpha-adrenergic antagonist drugs, B′6) beta-adrenergic agonist drugs, B′7) antidepressant drugs, B′8) GABA agonist drugs, B′9) agonist drugs of the muscarinic receptor, and B′10) other drugs selected from inaperizone (B′10b), moxonidine (B′10c), papaverine (B′10e), benzydamine (B′10g):

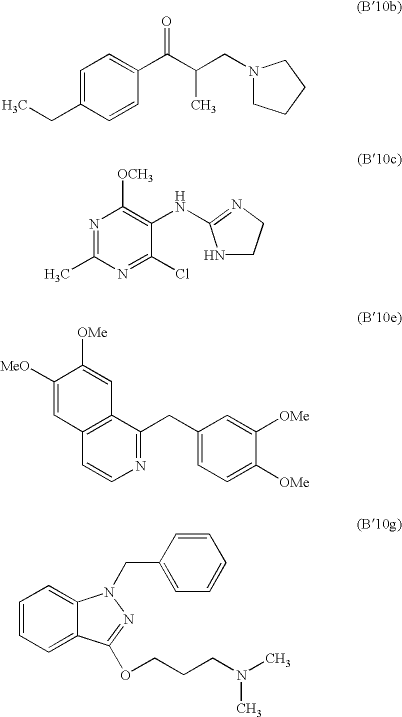

B11) serotoninergic antagonist drugs of the 5-HT₄ receptor.

In particular, compounds B′) are selected from the following:

B′1) propantheline (B′1a), emepronium (B′1b), trospium (B′1c), tolterodine (B′1d), dariphenacine (B′1e), vamicamide (B′1f), zamiphenacine (B′1g), atropine (B′1h), cyclodrine (B′1i), oxybutynin (B′1l), N-desethyl-oxybutynin (B′1l-I), dicyclomine (B′1m), propiverine (B′1n), flavoxate (B′1o), terodiline (B′1p);

B′2) nifedipine (B′2a), flunarizine (B′2b), diltiazem (B′2c);

B′3) pinacidil;

B′4) ephedrine (B′4a), pseudoephedrine, phenylpropanolamine (B′4c), midodrine (B′4d), de-glymidodrine (B′4e);

B′5) alfuzosin (B′5a), doxazosin (B5′b), prazozin (B′5c)

B′6) clenbuterol (B′6a), terbutaline (B′6b), formoterol (B′6c);

B′7) imipramine (B′7a), clozapine (B′7b), milnacipran (B′7c), fluphenazine (B′7d), nortriptyline (B′7e), duloxetine (B′7f);

B′8) baclofen;

B′9) bethanechol;

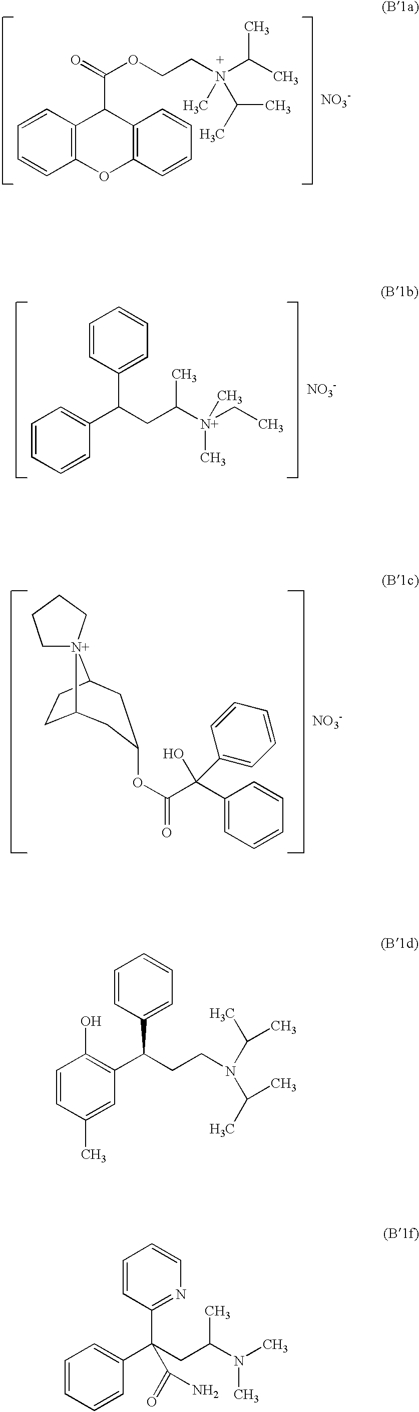

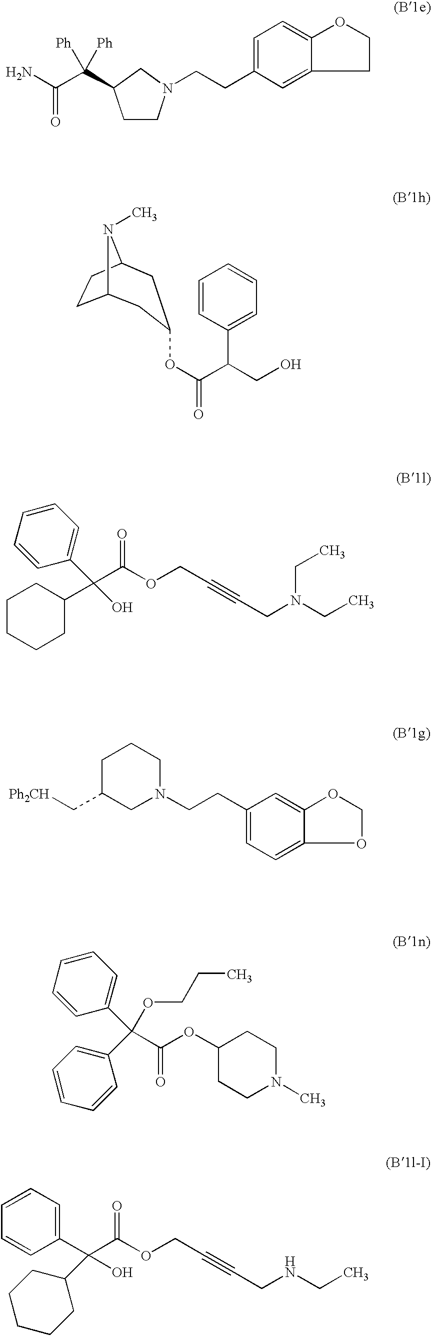

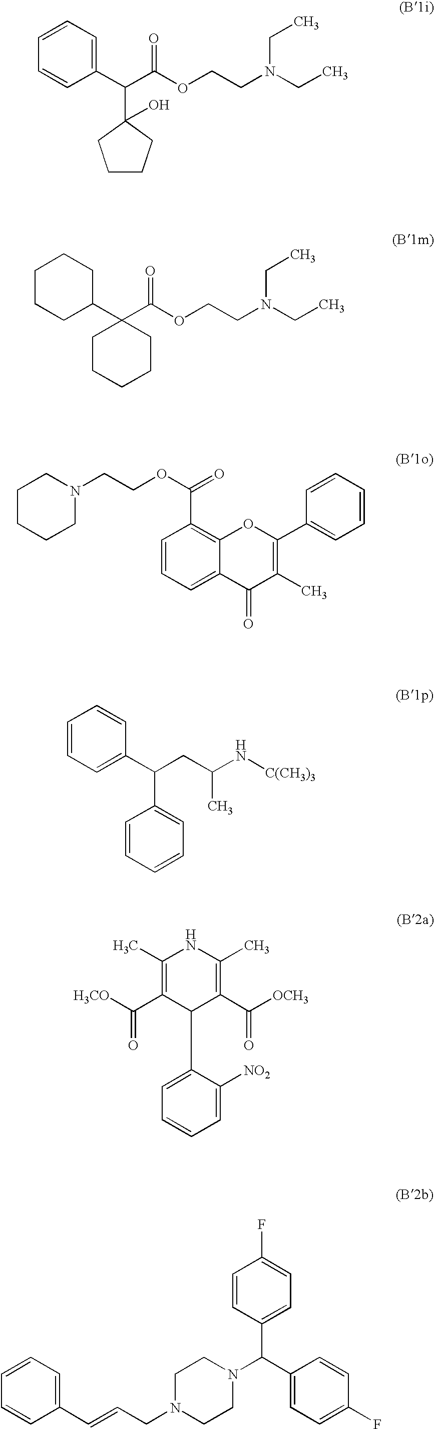

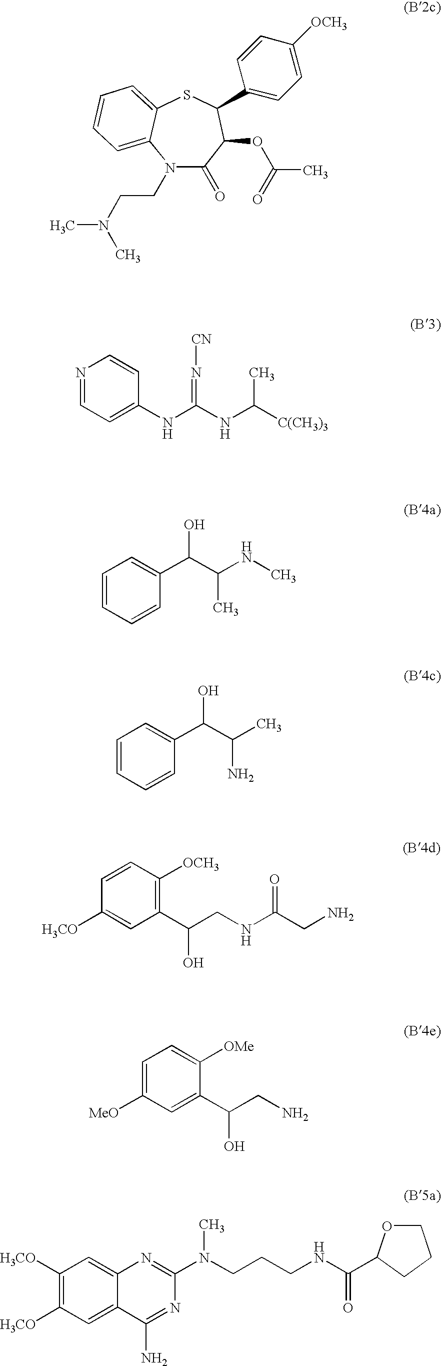

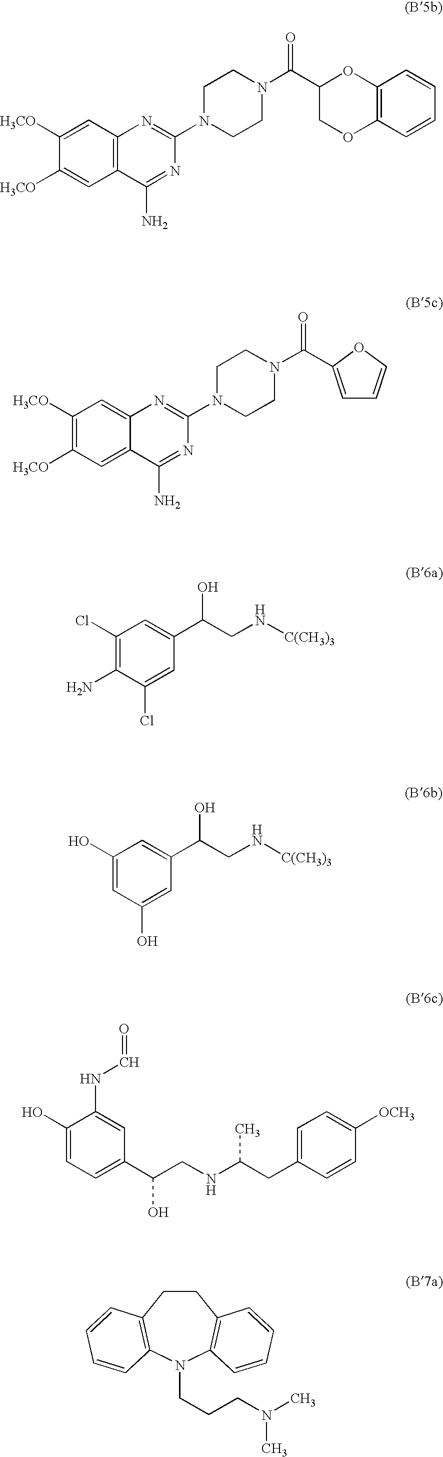

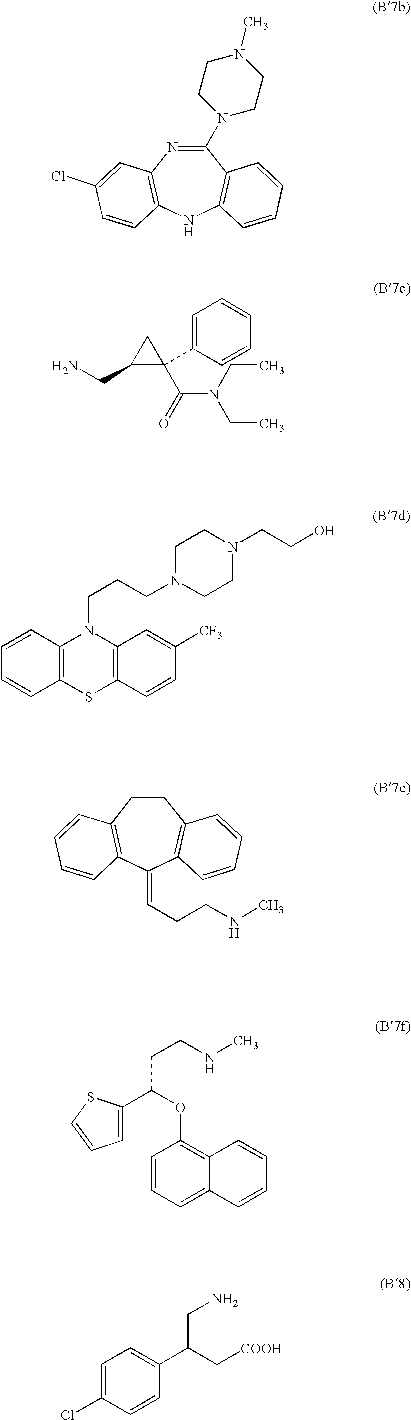

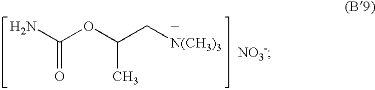

B′11) 3-(piperidin-1-yl)propyl 4 amino-5-chloro-2-methoxy benzoate (B′11a), 1-[4-amino-5-chloro-2-(3,5-dimethoxy phenyl)methyl oxy]-3-[1-[2-methylsulphonylamino]ethyl piperidin-4-yl]-1-propanone (B′11b), 2 (1-piperidinyl) ethyl-1H-indol-3-carboxylate (B′11c), (S)-2-chloro-5-methoxy-4-[5-(2-piperidylmethyl)-1,2,4-oxadiazol-3-yl]aniline (B′11d).

The synthesis of the compounds belonging to the classes B′1)-B′9) are described in the volume The Merck Index 12a Ed.; the synthesis of compound B′1e) is described in EP 388,054; of compound B′1g) is described in EP 350,309, of compound B′1d) is described in EP 325,571. The synthesis of compound B′11a) is carried out as described in EP 501,322, of compound B′11b) as described in Br J. Pharmacol. 1995, 115. 1087-1095, of compound B′11c) as described in EP 429,984.

The compounds inhibiting the phosphodiesterasr C) salifiable with organic or inorganic acids are selected from the following: (C1) 1-[4-ethoxy-3-(6,7-dihydro-1-methyl-7-oxo-3-propyl-1H-pyra-zol[4,3-d]-pyrimidin-5-yl)-phenyl] sulphoyl]-4-methyl-piperazine (Sildenafil), (C2) 2-(2-propyloxyphenyl)-8-azapurin-6-one (Zaprinast), (C3) 2,6-bis-(diethanolamino)-4,8-dipiperidine pyrimido [5,4-d]-pyrimidine (dipyridamol), (C4) 6-chloro-4-(1,3-dioxaindan-5-yl) methylamino-2 (4-carboxy-1-piperidinyl)-quinazoline, (CS)N-(phenylmethyl)-1-ethyl-1H-pyrazol-[3,4-b]-quinolin-4-amine, (C6) 1-(2-chlorobenzyl)-3-isobutyryl-2-propyl-6-aminocarbonyl-indol, (C7) 1-benzyl-6-chloro-2-[1-[3-(imidazol-1-il)propyl]indol-5-yl-amino carbonyl]benzimidazol, (C8) 2-(1-imidazolyl)-5-(phenyl)-4-(1,3-dioxaindan-5-yl)methyl aminopyrimidine, (C9) 6-ethynyl-4-(2-methoxyethyl)amino-2-(1-imidazolyl)quinazoline, (C10) 1-cyclopentyl-3-ethyl-6-(2-propoxyphenyl)pyrazol[3,4-d]pyrimidin-4-one, (C11) 1-cyclopentyl-3-ethyl-6-(4-methoxybenzyl)-pyrazol-[3,4-d]-pyrimidin-4-one, (C12) 1,3-dimethyl-6-(2-propoxy-5-methansulphonamidophenyl)-1,5-dihydro pyrazo1[3,4-d]-pyrimidin-4-one, (C13) (6R, 12aR)-2,3,6,7,12, 12a-hexahydro-2-methyl-6-(1,3-dioxan-5-yl)pyrazin [2′,1′:6,1]pyrido[3,4-b]indol-1,4-dione, (C14) 1-propyl-3-methyl-6-[2-propoxy-5-[(41-methyl-1-pyrazinyl)sulphonamido]phenyl]-1,5-dihydropyrazol[3,4-d]pyrimidin-4-one, (C15) 3-(4-amino carbonyl-1-piperidinyl)-6-cyan-8-(3-chloro-4-methoxy-phthalazine, (C16) 2-(1-imidazolyl)-4-(1,3-dioxaindan-5-yl) methylamino-7,8-dihydro-5H-thiopyran[3,2-d]pyrimidine, (C17) 1-Cyclopentyl-3-ethyl-6-(3-ethoxypyrid-4-yl)-1H-pyrazolo[3,4-d] pyrimidin-4-one, (C18) 1-[3-[1-((4-Fluorophenyl)methyl]-7,8-dihydro-8-oxo-1H-imidazo[4,S-g]quinazolin-6-yl]-4-propoxyphenyl]carboxamide.

Examples of organic salts of C) are oxalate, tartrate, maleate, succinate, citrate, glycinate, lysinate; examples of inorganic anions are nitrate, chloride, sulphate, phosphate. Nitrate salts are preferred.

The above compounds inhibiting the phosphodiesterases are sinthesized as described in the following references: (C1): G.B. 92,480; (C2): DE 2,162,096; (C3): The Merck Index 12th Ed.; (C4): WO 9422855; (C5): WO 9628159; (C6): WO 9632379; (C7): WO 9703070; (C8): U.S. Pat. No. 5,525,604; (C9): U.S. Pat. No. 5,436,233; (C10): WO 9628448; (C11): WO 9628429; (C12): EP 636,626; (C13): WO 9519978; (C14): EP 636,626; (C15): WO 9605176; (C16): EP 728,759; (C17): U.S. Pat. No. 5,294,612; (C18): J. Med. Chem 2000 43 1257-1263.

The nitrate salts of compounds B′) and of compounds C) can be prepared as for example described in patent application WO 99/45004 in the name of the Applicant; the other salts of compounds C) with anions different from nitrate are prepared by methods known in the prior art, such as for example described in patent application WO 96/28448.

For the use according to the present invention one or more salts of the drugs of classes A)-C) are formulated in the corresponding pharmaceutical formulations according to well known techniques in the art, together with the usual excipients. The formulations can be for oral, parenteral use and are prepared as known in the prior art. See for example the volume “Remington's Pharmaceutical Sciences 15th Ed.” The dosages of the salts of the invention in their pharmaceutical compositions are the same, and generally lower than those of their precursors of the above mentioned classes, said salts generally being more effective and better tolerated.

The following Examples illustrate but do not limit the scope of the invention.

### EXAMPLE 1

Preparation of Oxybutynin Nitrate Salt (B11) To a solution of oxybutynin chloride (1.1 g, 2.82 mmoles) (B′11) in acetonitrile (25 ml) silver nitrate (0.48 g, 2.82 mmoles) dissolved in acetonitrile (10 ml) is added. The mixture is maintained under stirring for 30 minutes sheltered from light and at room temperature. The precipitate (AgCl) is filtered and the solution is concentrated under reduced pressure up to half of the initial volume. Ethyl ether (50 ml) is added. By cooling with ice a precipitate is separated which is filtered and washed with ethyl ether (3×5 ml). After drying 0.8 g of oxybutynin nitrate salt are obtained as an amorphous solid. Yield 68%.

Melting point 86-87° C.

Elementary Analysis

| | | | |
|---|---|---|---|
| Calculated %: | C 62.84 | H 7.67 | N 6.66 |
| Found %: | C 62.67 | H 7.66 | N 6.70 |

### EXAMPLE 2

Preparation of Benzidamine Nitrate Salt (B′10g)

1) Preparation of Benzidamine Base

Benzidamine hydrochloride (3 g, 8.7 mmoles) (B′10g) is dissolved in an aqueous solution of sodium hydroxide (10% w/w, 45 ml) and the solution is extracted with ethyl acetate (3×50 ml). The joined organic phases are washed with water, anhydrified with sodium sulphate and the solvent evaporated under reduced pressure. An yellow oil formed by benzidamine free base is obtained.

¹H NMR. (DMSO): 7.65-7.6 (2H, m); 7.45-7.2 (6H, m); 7.15 (1H, t); 5.45 (2H, S); 4.4 (2 h, t); 2.4 (2H, t); 2.2 (6H, s); 2.0 (2H, m).

2) Preparation of Benzidamine Nitrate Salt

To a solution of benzidamine (2.5 g, 8.1 mmoles) in acetonitrile (15 ml), cooled at 0° C., nitric acid 65%k (0.560 ml, 8.1 mmoles) is added. The mixture is maintained under stirring at 0° C. for 30 minutes, the temperature is let reach the room temperature and the mixture is maintained under stirring for 1 hour. After addition of ethyl ether (10 ml) a white solid is separated which is filtered and washed with ethyl ether. After drying 2.6 g of benzidamine nitrate salt are obtained. Melting point 143-144° C.

Elementary Analysis

| | | | |
|---|---|---|---|
| Calculated %: | C 61.29 | H 6.49 | N 15.04 |
| Found %: | C 60.93 | H 6.45 | N 14.97 |

### EXAMPLE 3

Preparation of Papaverine Nitrate Salt (B′10e)

1) Preparation Papaverine Base

Papaverine hydrochloride (3 g, 8 mmoles) (B′10e) is dissolved in an aqueous solution of sodium hydroxide (10% w/w, 50 ml) and the solution is extracted with chloroform (3×50 ml). The joined organic phases are washed with water, anhydrified with sodium sulphate and the organic solvent evaporated under reduced pressure. Papaverine base (2.7 g) is obtained as an amorphous solid.

¹H NMR (DMSO): 8.4 (1H, d); 7.6 (2H, d); 7.4 (1H, s); 6.8 (2H, m); 4.5 (2H, s); 3.9 (6H, d); 3.7 (6H, d)

2) Preparation of Papaverine Nitrate Salt

To a solution of papaverine (2.6 g, 7.6 mmoles) in acetonitrile (100 ml), cooled at 0° C., nitric acid 65% (0.560 ml, 8.1 mmoles) is added. The mixture is maintained under stirring at 0° C. for 30 minutes, it is let reach the room temperature and the mixture is maintained under stirring for 2 hours. The formed precipitate is filtered and washed with acetonitrile. After drying 2.3 g of papaverine nitrate salt are obtained.

Elementary Analysis

| | | | |
|---|---|---|---|
| Calculated %: | C 59.69 | H 5.51 | N 6.96 |
| Found %: | C 58.68 | H 5.38 | N 6.86 |

### EXAMPLE 4

Preparation of Phenylpropanolamine Nitrate Salt (B′4c)

To a solution of phenylpropanolamine hydrochloride (2 g, 10.75 mmoles) (B′4c) in acetonitrile (50 ml) silver nitrate (1.83 g, 10.77 mmoles) is added. The salt preparation is carried out following the procedure described in Example 1. The phenylpropanolamine nitrate salt is obtained as an amorphous solid.

Elementary Analysis

| | | | |
|---|---|---|---|
| Calculated %: | C 50.46 | H 6.59 | N 13.08 |
| Found %: | C 50.60 | H 6.62 | N 13.12 |

### EXAMPLE 5

Preparation of Pinacidil Nitrate Salt (B′3)

To a solution of pinacidil (3 g, 12.23 mmoles) (B′3) in acetonitrile (100 ml), cooled at 0° C., nitric acid 65% (0.850 ml, 12.27 mmoles) is added. The mixture is maintained under stirring at 0° C. for 30 minutes. At the end it is let reach the room temperature and the mixture is maintained under stirring for 2 hours. After addition of ethyl ether a precipitate forms which is filtered and washed with ethyl ether (3×20 ml). After drying 2.3 g of pinacidil nitrate salt are obtained. Yield 60%.

Elementary Analysis

| | | | |
|---|---|---|---|
| Calculated %: | C 50.64 | H 6.54 | N 27.26 |
| Found %: | C 50.73 | H 6.62 | N 27.19 |

### EXAMPLE 6

Preparation of Terodiline Nitrate Salt (B′1p)

Terodiline hydrochloride (2 g, 6.3 mmoles) (B′1p) is dissolved in an aqueous solution of sodium hydroxide (10% w/w, 35 ml) and the solution extracted with ethyl acetate (3×50 ml). The joined organic phases are washed with water, anhydrified with sodium sulphate and the organic solvent evaporated under reduced pressure. The residue is dissolved in acetonitrile (15 ml) and the solution is cooled at 0° C. Nitric acid 65% (0.440 ml, 6.35 mmoles) is added. The mixture is maintained under stirring at 0° C. for 30 minutes, then it is let reach the room temperature and the mixture is maintained under stirring for 1 hour. By adding ethyl ether (10 ml) a white solid is separated which is filtered and washed with ethyl ether. After drying 1.2 g of terodiline nitrate salt are obtained. Yield 55%.

Elementary Analysis

| | | | |
|---|---|---|---|
| Calculated %: | C 69.74 | H 8.19 | N 8.13 |
| Found %: | C 69.80 | H 8.25 | N 8.09 |

### EXAMPLE 7

Preparation of Propantheline Nitrate Salt (B′1a)

The compound is prepared starting from a solution of propantheline bromide (3 g, 6.7 mmoles) (B′1a) in acetonitrile (80 ml), adding silver nitrate (1.3 g, 7.06 mmoles) dissolved in acetonitrile (10 ml), and following the procedure described in Example 1. After drying, propantheline nitrate salt is obtained as an amorphous solid (1.4 g). Yield 486.

Elementary Analysis

| | | | |
|---|---|---|---|
| Calculated %: | C 64.02 | H 7.24 | N 6.49 |
| Found %: | C 64.11 | H 7.27 | N 6.45 |

### EXAMPLE 8

Preparation of Flavoxate Nitrate Salt (B′10)

The compound is prepared starting from a solution of flavoxate hydrochloride (2 g, 4.7 mmoles) (B′1o) in acetonitrile (50 ml) adding a silver nitrate solution (0.800 g, 4.76 mmoles) in acetonitrile (10 ml) and following the procedure described in Example 1. After drying flavoxate nitrate salt is obtained as an amorphous solid (1.1 g). Yield 50%.

Elementary Analysis

| | | | |
|---|---|---|---|
| Calculated %: | C 63.42 | H 5.77 | N 6.16 |
| Found %: | C 63.52 | H 5.98 | N 6.20 |

### EXAMPLE 9

Preparation of Dicyclomine Nitrate Salt (B′1m)

The compound is prepared starting from a solution of dicyclomine hydrochloride (2 g, 5.78 mmoles) (B′1m) in acetonitrile (50 ml) adding silver nitrate (0.990 g, 4.76 mmoles) dissolved in acetonitrile (10 ml) and following the procedure described in Example 1. After drying dicyclomine nitrate salt is obtained as an amorphous solid (1.3 g). Yield 60%.

Elementary Analysis

| | | | |
|---|---|---|---|
| Calculated %: | C 61.26 | H 9.74 | N 7.52 |
| Found %: | C 61.19 | H 9.69 | N 7.58 |

### PHARMACOLOGICAL EXAMPLES

The activity in the urinary incontinence of the compounds according to the present invention has been evaluated in an experimental model of inhibition of the bladder contraction.

The degree of the relaxation induced in the urinary bladder is a measure of the inhibitory action of the urinary incontinence of the drugs described in the present application.

Guinea-pigs of male sex having an average weight equal to 300-500 g were sacrificed and bled. The urinary bladder was removed and prepared for determining the myorelaxing activity in vitro, according to the method described by L. Nilvenbrant, Eur. J. Pharmacol. 327,195-207, 1997.

The obtained tissue strips were contracted with carbacol 10⁻⁶ M in phisiological solution and the relaxation was determined in the presence of the compounds indicated in Table 1 at the concentrations mentioned therein. The 2-(acetyloxy) benzoic acid 6-(nitroxymethyl)-2-methylpyridyl ester hydrochloride was prepared according to Example 1 of patent application PCT/EP 00/01454 (NCX 4050).

The sildenafil nitrate salt was prepared as described in patent application WO 99/67231 (Ex. 3).

In Table 1 the results are expressed as a percentage of the maximum inhibition of the contractions induced by carbacol 10⁻⁶ M and they show that the compounds of the present invention are active in the urinary incontinence since they are able to exert a significant relaxing effect on the urinary guinea-pig bladder, which in the case of the nitrate salts of the drugs used in the incontinence is higher than that of the precursors.

TABLE 1
Activity in the urinary incontinence of the
compounds
NCX 4050, sildenafil nitrate, sildenafil
citrate, oxybutynin nitrate and oxybutynin
hydrochloride in an experimental model in vitro of
strips of urinary guinea-pig bladder contracted
with carbacol

| | Concentration | Urinary bladder |
|---|---|---|
| Treatment | (M) | concentration % |
| Placebo | — | 100 |
| NCX 4050 | 3 × 10 − 5 | 71 |
| Sildenafil | 3 × 10 − 5 | 31 |
| Nitrate | | |
| Sildenafil | 3 × 10 − 5 | 52 |
| Citrate | | |
| Oxybutynin | 10⁻⁶ | 30 |
| Hydrochloride | | |
| (Comp) | | |
| Oxybutynin | 10⁻⁶ | 0 |
| Nitrate | | |

1. Use in the incontinence of one or more of the following classes of drugs selected from the following:
A) nitric oxide donor drugs, salified and non salified of formula
A-X₁—N(O)_{z}
wherein A, X₁, Z have the meaning defined below;
B′) nitrate salts of drugs used for the incontinence and which do not contain in the molecule a nitric oxide donor group;
C) organic or inorganic salts of compounds inhibiting phosphodiesterases;
in the compounds of general formula:
A-X₁—N(O)_{z}
z is an integer and is 1 or 2, preferably 2;
A=R(COXᵤ)ₜ and wherein t is an integer 0 or 1; u is 0 or 1;
X=O, NH, NR_{1c} wherein R_{1c} is a linear or branched C₁-C₁₀ alkyl;
X₁ is the following bivalent linking group: 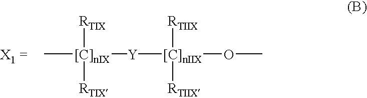
wherein:
nIX is an integer in the range 0-3;
nIIX is an integer in the range 1-3;
R_{TIX}, R_{TIX′}, R_{TIIX}, R_{TIIX′}, equal to or different from each other are H or a linear or branched C₁-C₄ alkyl;
Y is a heterocyclic ring containing one or two nitrogen atoms, optionally one oxygen or sulphur atom, said saturated, unsaturated or aromatic ring having 5 or 6 atoms;
R of the radical A of formula A-X₁—N(O)_{z} is selected from the following groups:
Group I) wherein t=1 and u 1 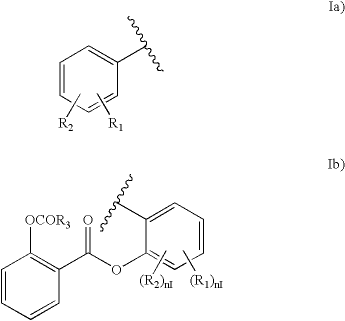
wherein:
R₁ is the OCOR₃ group; wherein R₃ is methyl, ethyl or a linear or branched C₃-C₅ alkyl, or the residue of a heterocycle having only one ring having 5 or 6 atoms which can be aromatic, partially or totally hydrogenated, containing one or more heteroatoms independently selected from O, N and S;
R₂ is hydrogen, hydroxy, halogen, linear or branched C₁-C₄ alkyl, linear or branched C₁-C₄ alkoxy; a linear or branched C₁-C₄ perfluoroalkyl, for example trifluoromethyl; nitro, amino, mono- or di-(C₁₋₄) alkylamino;
nI is an integer 0 or 1;
group II) wherein t=1, u=1 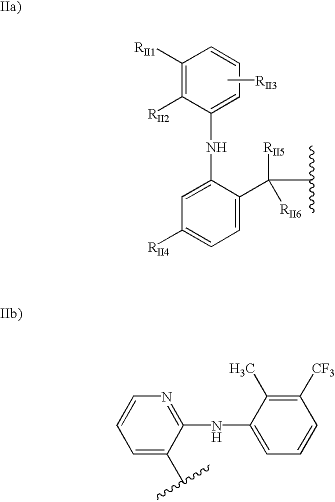
wherein:
R_{II5} is H, linear or branched when possible C₁-C₃ alkyl;
R_{II6} has the same meaning as R_{II5}, or when R_{II5} is H it can be benzyl;
R_{II1}, R_{II2} and R_{II3} can independently be hydrogen, linear or branched when possible C₁-C₆ alkyl, or linear or branched when possible C₁-C₆ alkoxy, or Cl, F, Br;
R_{II4} is R_{II1} or bromine;
IIb) is the residue of the 2-[(2-methyl-3-(trifluoromethyl)phenyl]amino]-3-pyridincarboxylic] acid and when the —COOH group is present the compound is known as flunixin;
group III) wherein t=1, u=1 and R is 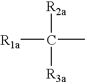
wherein:
R₂ₐ and R₃ₐ are H, linear or branched when possible, substituted or not, C₁-C₁₂ alkyl or allyl, with the proviso that if one of the two is allyl the other is H; preferably R₂ₐ is H, C₁-C₄ alkyl, R₃ₐ is H;
R₁ₐ is selected from 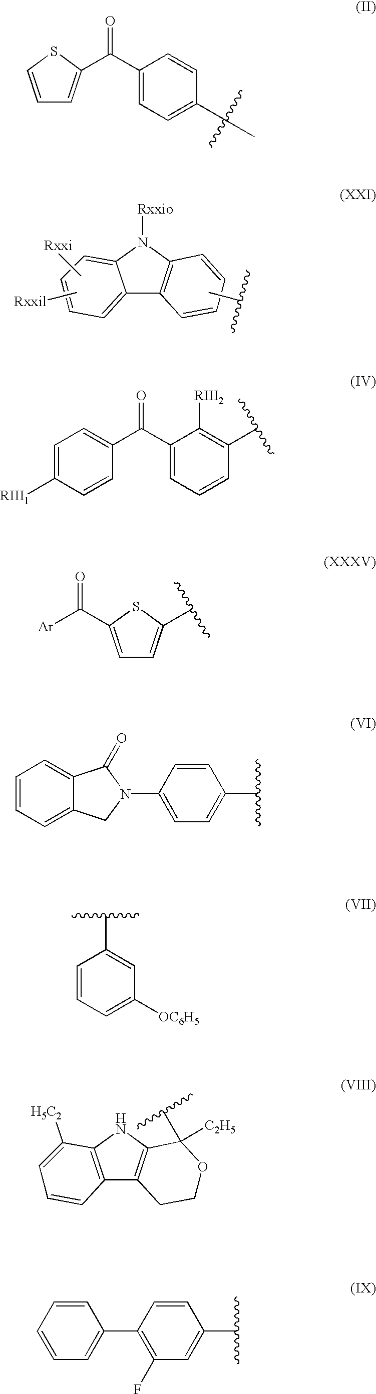 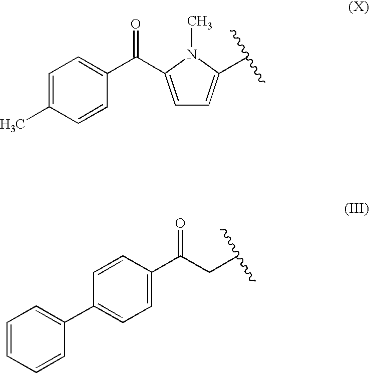
IIID) R₁ₐ corresponds to the following formulas: 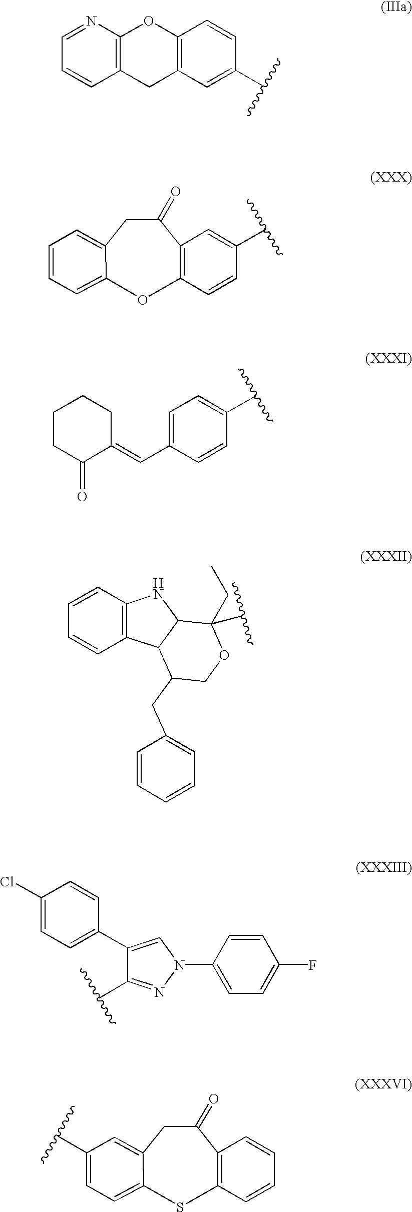 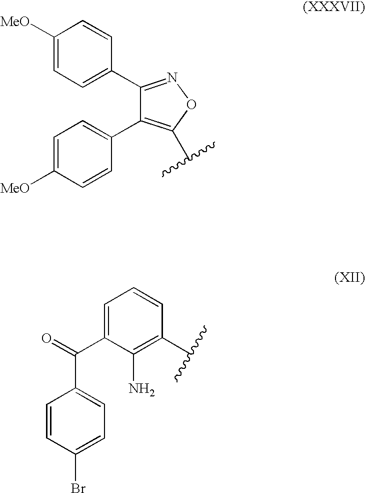
wherein the meanings are the following:
when R₁ₐ is as defined in formula (IV), Ketoprofen residue: R_{III1} is H, SR_{III3} wherein R_{III3} contains from 1 to 4 carbon atoms, linear or branched when possible; R_{III2} is H, hydroxy;
when R₁ₐ is as defined in formula (XXI), carprofen residue: Rₓₓᵢₒ is H, linear or branched when possible alkyl from 1 to 6 carbon atoms, C₁-C₆ alkoxycarbonyl linked to a C₁-C₆ alkyl, C₁-C₆ carboxyalkyl, C₁-C₆ alkanoyl, optionally substituted with halogens, benzyl or halobenzyl, benzoyl or halobenzoyl;
Rₓₓᵢ is H, halogen, hydroxy, CN, C₁-C₆ alkyl optionally containing OH groups, C₁-C₆ alkoxy, acetyl, benzyloxy, SRₓₓᵢ₂ wherein Rₓₓᵢ₂ is C₁-C₆ alkyl; C₁-C₃ perfluoroalkyl; C₁-C₆ carboxyalkyl optionally containing OH groups, NO₂, amino; sulphamoyl, di-alkyl sulphamoyl with C₁-C₆ alkyl, or difluoroalkylsulphonyl with C₁-C₃ alkyl;
Rₓₓᵢ₁ is halogen, CN, C₁-C₆ alkyl containing one or more OH groups, C₁-C₆ alkoxy, acetyl, acetamido, benzyloxy, SR_{III3}, being R_{III3} as above defined, C₁-C₃ perfluoroalkyl, hydroxy, C₁-C₆ carboxyalkyl, NO₂, amino, mono- or di-alkyl-amino C₁-C₆; sulphamoyl, di-alkyl sulphamoyl C₁-C₆, or di-fluoroalkylsulphamoyl as above defined; or Rₓₓᵢ together with Rₓₓᵢ₁ is a C₁-C₆ alkylen dioxy;
when R₁ₐ is as defined in formula (XXXV) tiaprofenic acid residue:
Ar is phenyl, hydroxyphenyl optionally mono or polysubstituted with halogen, alkanoyl and alkoxy C₁-C₆, C₁-C₆, preferably C₁-C₃, trialkyl, cyclopentyl, cyclohexyl, cycloheptyl, heteroaryl, preferably thienyl, furyl optionally containing OH, pyridyl;
when R₁ₐ is as defined in formula (II), suprofen residue, wherein R₃ₐ is H, R₂ₐ is methyl and X=O;
when R₃ₐ is as defined in formula (VI), R is the residue of indoprofen when R₂ₐ=H and R₃ₐ=CH₃; of indobufen when R₂ₐ is equal to H and R₃ₐ=C₃H₅; X=O;
when R₁ₐ is as defined in formula (VIII), R is the etodolac residue when R₂ₐ=R₃ₐ=H and X=O;
when R₁ₐ is as defined in formula (VII), R is the fenoprofen residue when R₃ₐ=H, R₂ₐ=CH₃ and X=O;
when R₁ₐ is as defined in formula (III), R is the fenbufen residue when R₂ₐ=R₃ₐ=H and X=O;
when R₁ₐ is as defined in formula (IX), R is the flurbiprofen residue when R₃ₐ═H, R₂ₐ=CH₃, X=O;
when R₁ₐ is as defined in formula (X) R is the tolmetin residue when R₂ₐ=R₃ₐ=H, X=O;
in group IIID) R₁ₐ corresponds to the following formulas:
IIIa), when R₂ₐ=H and R₃ₐ=CH₃ the pranoprofen residue is obtained: α-methyl-5H-[1]benzopyran-[2,3-b]pyridin-7-acetic acid; the preferred compond has R₂ₐ=H, R₃ₐ=CH₃, u=1 and X=0;
(XXX), when R₂ₐ=H and R₃ₐ=CH₃ the bermoprofen residue is obtained: dibenz[b,f]oxepin-2-acetic acid;
(XXXI), when R₂ₐ=H and R₃ₐ=CH₃, R is the radical of the CS-670 compound: 2-[4-(2-oxo-1-cyclohexyliden methyl) phenyl]propionic acid;
(XXXII), when R₂ₐ=R₃ₐ=H the Pemedolac residue is obtained;
(XXXIII), when R₂ₐ=R₃ₐ=H the pyrazolac residue is obtained: 4-(4-chlorophenyl)-1-(4-fluorophe-nyl)-3-pyrazolic acid;
(XXXVI), when R₂ₐ=H, R₃=CH₃ the zaltoprofen residue is obtained; when the residue is saturated with an hydroxyl or amino group, or with the carboxylic function the compounds are known as dibenzothiepine derivatives;
(XXXVII), when R₂ₐ=R₃ₐ=H the mofezolac residue is obtained: 3,4-di(p-methoxyphenyl)isoxazol-5-acetic acid;
(XII), when R₂ₐ=R₃ₐ=H the bromfenac residue is obtained: 2-amino-3-(4-bromobenzoyl)benzeneacetic acid;
in group IV) wherein t=1, u=1, R is 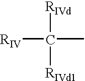
wherein:
R_{IVd} and R_{IVd1} are at least one H and the other a linear or branched C₁-C₆, preferably C₁ and C₂ alkyl, or difluoroalkyl with the alkyl from 1 to 6 carbon atoms, C₁ is preferred, or R_{IVd} and R_{IVd1} form together a methylene group;
R_{IV} has the following meaning: 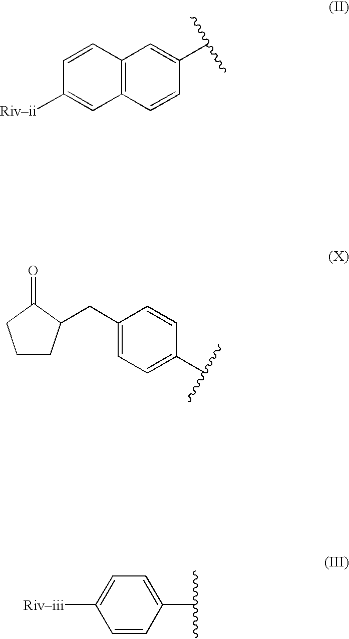
wherein the compounds of group IV) have the following meanings:
in formula (II):
R_{iV-ii} is C₁-C₆ alkyl, C₃-C₇ cycloalkyl, C₁-C₇ alkoxymethyl, C₁-C₃ trifluoroalkyl, vinyl, ethynyl, halogen, C₁-C₆ alkoxy, difluoroalkoxy, with the C₁-C₇ alkyl, C₁-C₇ alkoxymethyloxy, alkylthio methy-loxy with the C₁-C₇ alkyl, alkyl methylthio with the C₁-C₇ alkyl, cyan, difluoromethylthio, phenyl- or phenylalkyl substituted with C₁-C₅ alkyl.
formula (X) loxoprofen residue;
in formula (III):
R_{iV-iii} is a C₂-C₅ alkyl, optionally branched when possible, C₂ and C₃ alkyloxy, allyloxy, phenoxy, phenylthio, cycloalkyl from 5 to 7 carbon atoms, optionally substituted in position 1 by a C₁-C₂ alkyl; 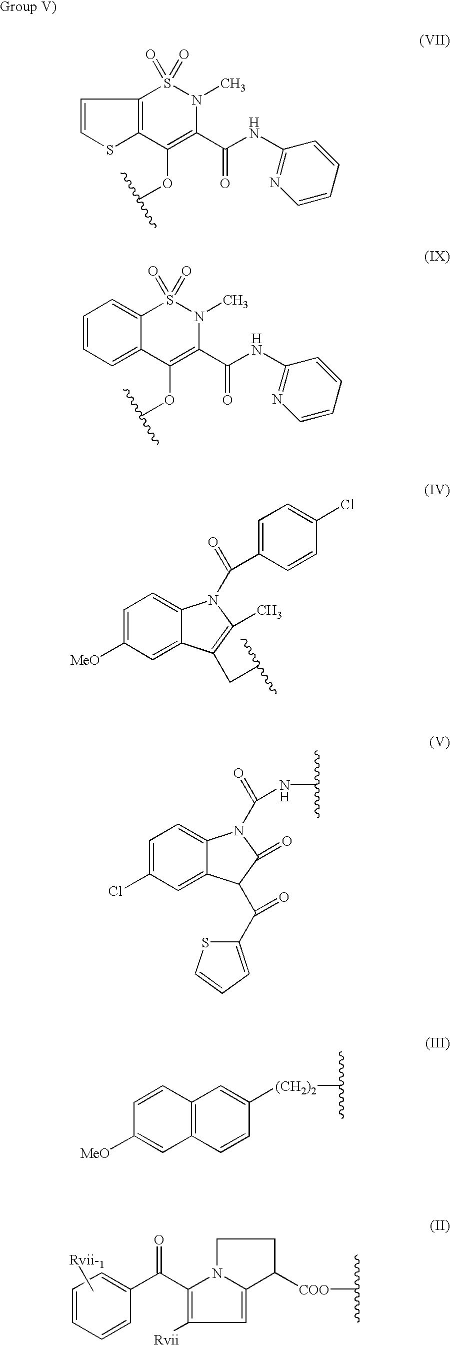 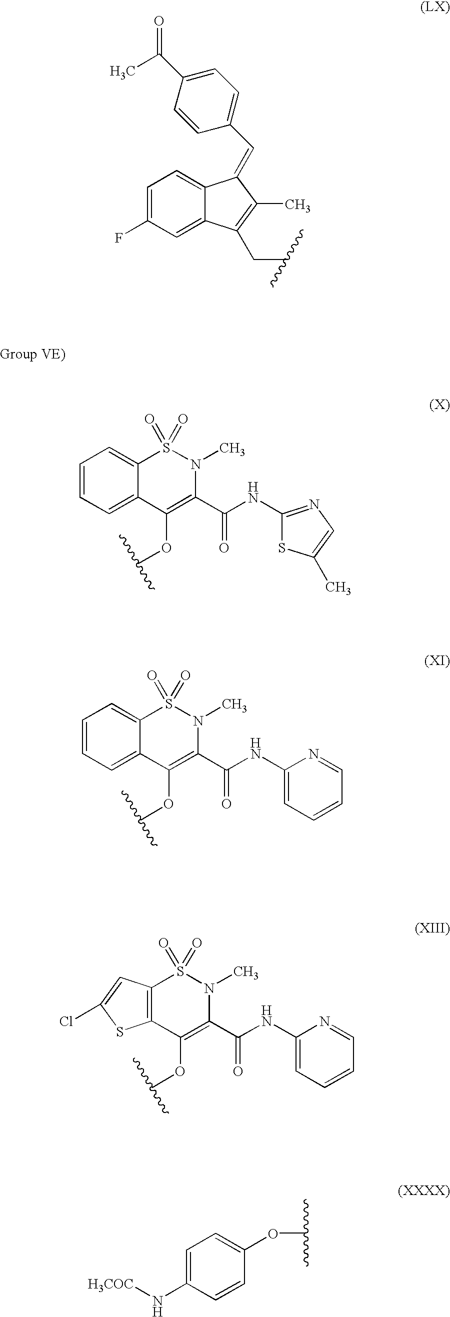 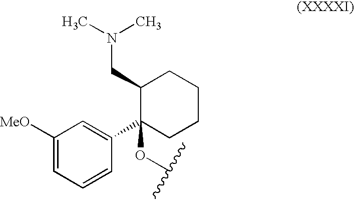
In group V):
when R is formula (II), R_{Vii} is H or a linear or branched C₁-C₄ alkyl;
R_{Vii-1} is R_{Vii}, or a linear or branched C₁-C₄ alkoxy; Cl, F, Br; the position of R_{Vii-1} being ortho, or metha, or para;
when R is formula (V),
of which the residue of the known tenidap has been indicated;
when R is formula (V) A=R and t=0,
when R is formula (VII), A is RCO, t=1 u=0 or A is R and t=0;
when R is formula (IX), A R and t=0, or A=RCO with t=1 and u=0;
when R is formula (III) A=RCOO, t=1 and u=0 or 1; or t=0 and A=R;
when R is formula (IV) A=RCOO, t=1 and u=1;
when R is formula (LX) and in (COXᵤ)ₜ u=t=1 and X is oxygen, the precursor compound is sulindac;
when R is formula (X) it is the meloxicam residue;
when R is formula (XI) the residue is known as ampiroxicam when the termination is —CH(CH₃)OCOC₂H₅;
when R is formula (XIII) and the valence is saturated with H, the residue derives from lornoxicam;
when R is formula (XXXX) and the valence is saturated with H the compound is known as paracetamol;
when R is formula (XXXXI) and the valence is saturated with H the compound is known as tramadol.

2. Use according to claim 1, wherein Y is selected from the following:

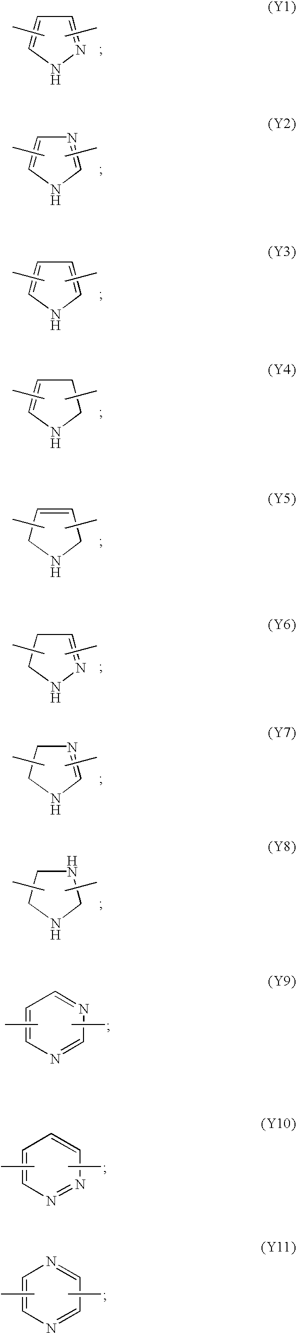

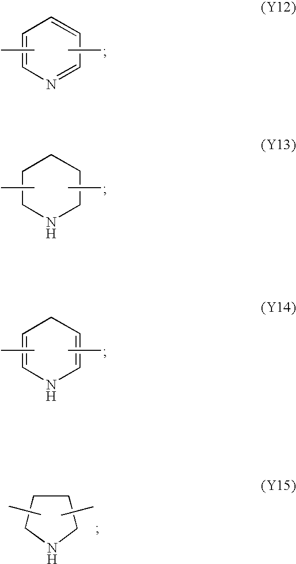

3. Use according to claim 2, wherein Y is Y12 (pyridyl) substituted in position 2 and 6.

4. Use according to claims 1-3, wherein in the compounds A) of formula A-X₁—N(O)_{z} is 2 and nIX and nIIX in formula (B) of X₁ are integers equal to 1 and R_{TIX}, R_{TIX′}, R_{TIIX}, R_{TIIX′} are equal to H.

5. Use according to claims 1-4, wherein in the compounds of formula A) A-X₁—N(O)_{z} R, X, u and t of formula A=R(COXᵤ)ₜ, and Y in formula (B) of X₁, have the following meanings:
when R is selected from group I), in the compounds of formula Ia) X is equal to O or NH, R₁ is acetoxy, preferably in ortho position with respect to —CO—, R₂ is hydrogen; in X₁ R_{TIX}=R_{TIX′}=R_{TIIX}=R_{TIIX}′=H, n_{IX}=n_{IIX}=1 and Y is an aromatic ring having 6 atoms, containing one nitrogen atom, said aromatic ring having the two free valences in position 2 and 6; in the compounds of formula Ib) R₃=CH₃, nI=0, X is equal to O, X₁ is as above defined for Ia); in this case Ib) is the residue of the acetylsalicylsalicylic acid;
when R is selected in group II) in formula IIa R_{II1}, R_{II4} are hydrogen and R_{II2} and R_{II3} are chlorine in ortho position with respect to NH; R_{II5} and R_{II6} are H, X is equal to O, and X₁ is as above defined for the compounds of formula Ia);
when R is selected in group III),
when R₁ₐ is as defined in formula (IV) R_{III1} and R_{III2} are H, R₃ₐ is H, and R₂ₐ is methyl, X=O;
when R₁ₐ is as defined in formula (XXI) Rₓₓᵢₒ is H, the linking group is in position 2, Rₓₓᵢ is H, Rₓₓᵢ₁ is chlorine and it is in para position with respect to nitrogen;
when R₁ₐ is as defined in formula (XXXV) Ar is phenyl, R₃ₐ is H, R₂ₐ is methyl and X is O; R₃ₐ is H, R₂ₐ is methyl and X is O;
when R₁ₐ is as defined in formula IIIa), R₂ₐ=H, R₃ₐ=CH₃, u=1 and X=O.
when R₁ₐ is as defined in formula (XXX) R₂ₐ=H, R₃ₐ=CH₃, u=1 and X=O;
when R₁ₐ is as defined in formula (XXXI), R₂ₐ=H, R₃ₐ=CH₃, u=1 and X=O;
when R₁ₐ is as defined in formula (XXXII), R₂ₐ=R₃ₐ=H, u=1 and X=O;
when R₁ₐ is as defined in formula (XXXIII), R₂ₐ=R₃ₐ=H, u=1 and X=O;
when R₁ₐ is as defined in formula (XXXVI), R₂ₐ=H, R₃ₐ=CH₃, u=1 and X=O;
when R₁ₐ is as defined in formula (XXXVII), R₂ₐ=R₃ₐ=H, t=1 and X=O;
when R₁ₐ is as defined in formula (XII), R₂ₐ=R₃ₐ=H, u=1, t=1, X=O, R₂ₐ=R₃ₐ=H; or t=0;
when R is selected in group IV),
when R_{IV} is the formula (II), R_{iV-ii}=CH₃O—, R_{IVd}=H and R_{IVd1}=CH₃, X=O and X₁ is as above defined for Ia);
when R_{IV} is formula (X), R_{IVd}=H, R_{IVd1}=CH₃, X=O and X₁ is as above defined for Ia);
when R_{IV} is formula (III), R_{iV-iii} is 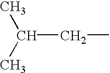
and R_{IVd}=H, R_{IVd1} is CH₃, X=O and X₁ is as above defined for Ia);
when R is selected in group V,
when R is formula (II), R_{Vii} and R_{Vii-1} are H, and A=R;
when R is formula (X), A=RCO, t=1 and u=0;
when R is formula (XI), A=RCO, t=1 and u=0;
when R is formula (XIII), A=RCO, t=1 and u=0;
when R corresponds to formula (XXXX) or (XXXXI), A=RCO, t=1 and u=0.

6. Use according to claims 1-5, wherein the drugs of the nitrate salts compounds B′) are selected from B′1) anticholinergic drugs, B′2) calcium antagonist drugs, B′3) drugs which facilitate the opening of the potassium channels, B′4) alpha-adrenergic agonist drugs, B′5) alpha-adrenergic antagonist drugs, B′6) beta-adrenergic agonist drugs, B′7) antidepressant drugs, B′8) GABA agonist drugs, B′9) agonist drugs of the muscarinic receptor and B′10) other drugs selected from inaperizone (B′10b), moxonidine (B′10c), papaverine (B′10e), benzydamine (B′10g) 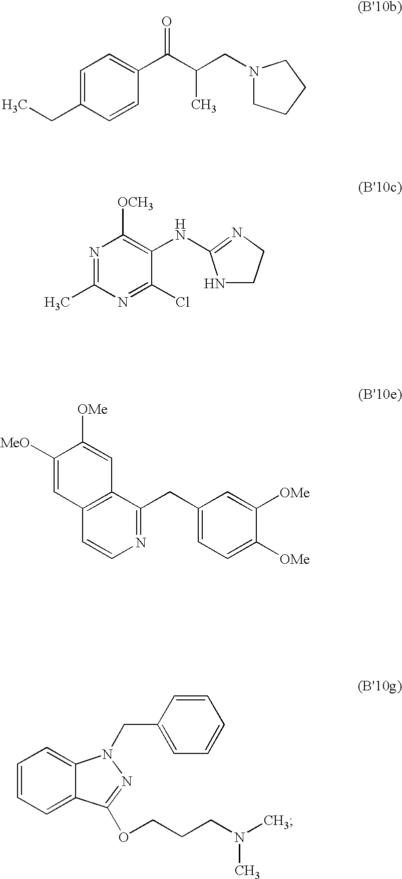
B11) antagonist serotoninergic drugs of the 5-HT₄ receptor.

7. Use according to claim 6, wherein the compounds B′) are selected from the following:
B′1) propantheline (B′1a), emepronium (B′1b), trospium (B′1c), tolterodine (B′1d), dariphenacine (B′1e), vamicamide (B′1f), zamiphenacine (B′1g), atropine (B′1h), cyclodrine (B′1i), oxybutynin (B′1l), N-desethyl-oxybutynin (B′1l-I), dicyclomine (B′1m), propiverine (B′1n), flavoxate (B′1o), terodiline (B′1p);
B′2) nifedipine (B′2a), flunarizine (B′2b), diltiazem (B′2c);
B′3) pinacidil;
B′4) ephedrine (B′4a), pseudoephedrine, phenylpropanolamine (B′4c), midodrine (B′4d), de-glymidodrine (B′4e);
B′5) alfuzosin (B′5a), doxazosin (B5′b), prazozin (B′5c)
B′6) clenbuterol (B′6a), terbutaline (B′6b), formoterol (B′6c);
B′7) imipramine (B′7a), clozapine (B′7b), milnacipran (B′7c), fluphenazine (B′7d), nortriptyline (B′7e), duloxetine (B′7f);
B′8) baclofen;
B′9) bethanechol; 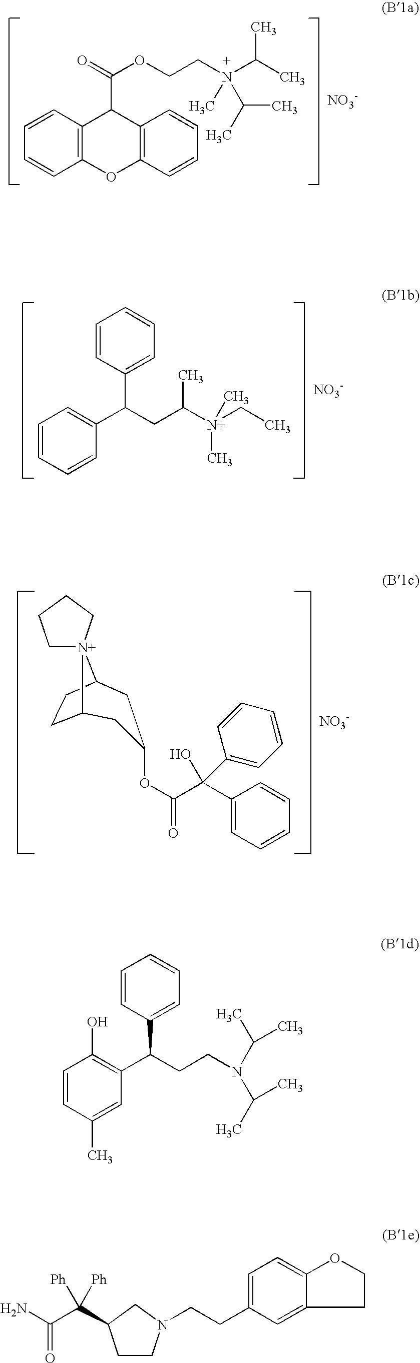 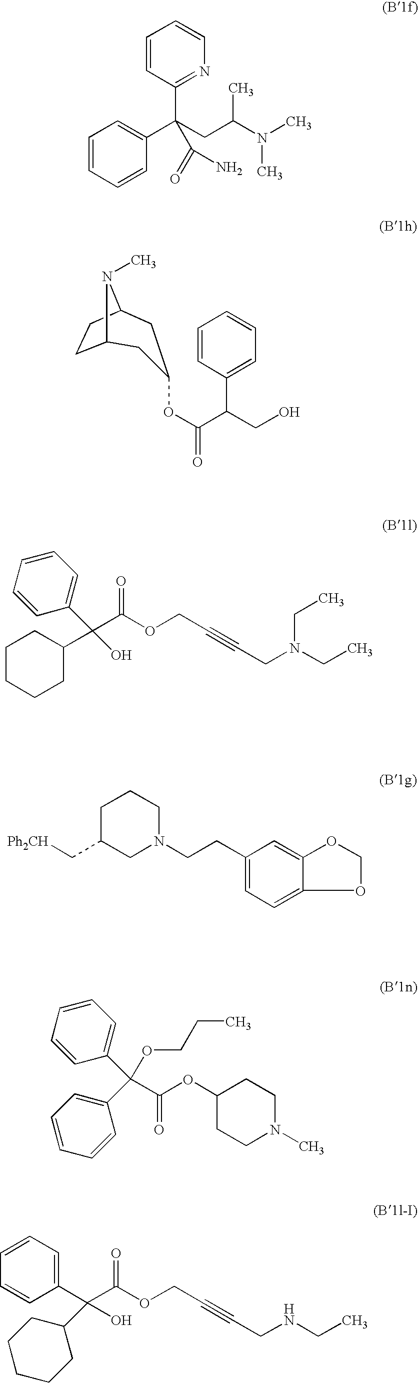 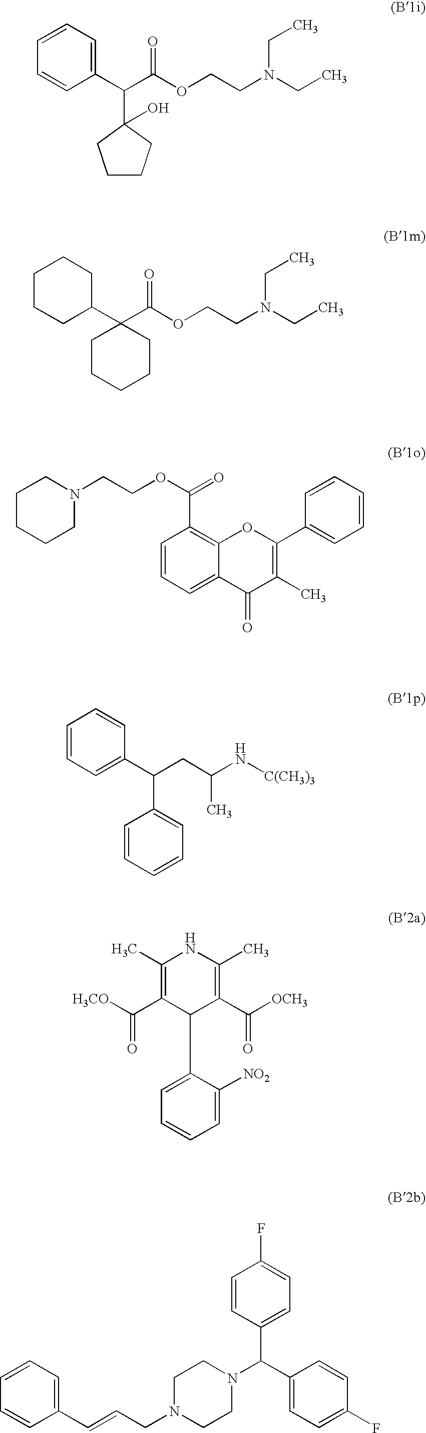 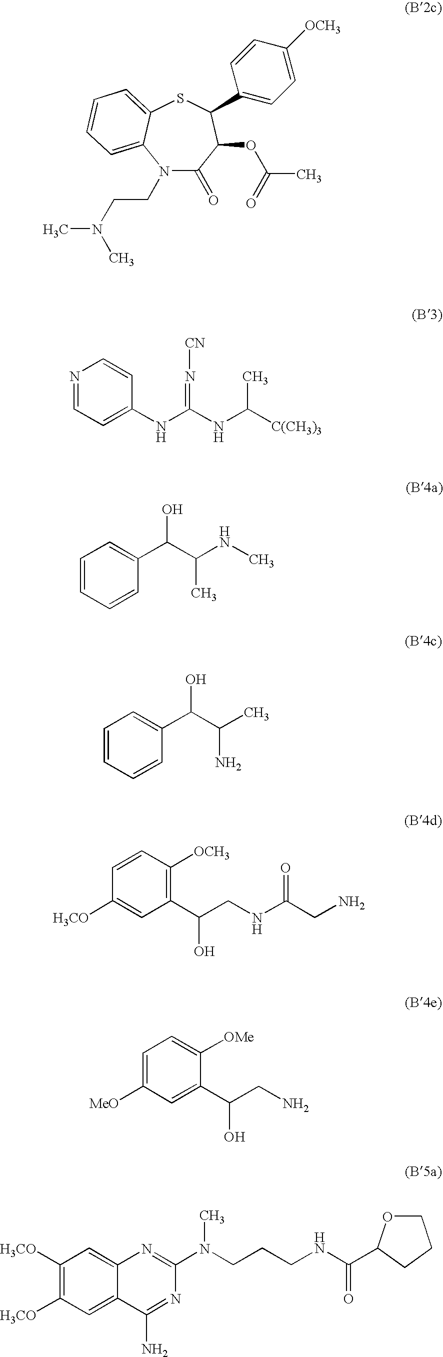 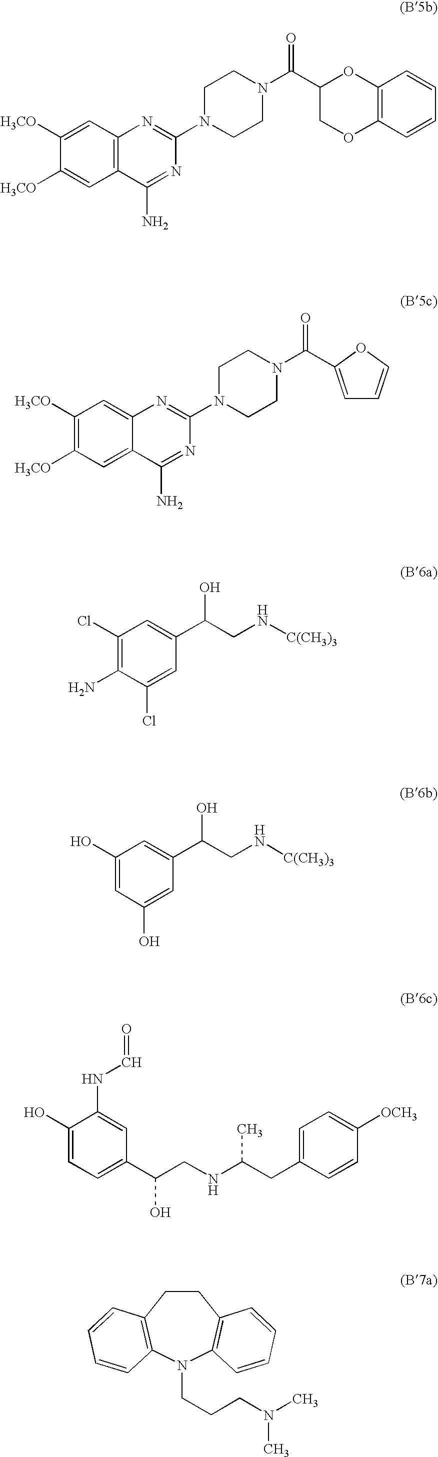 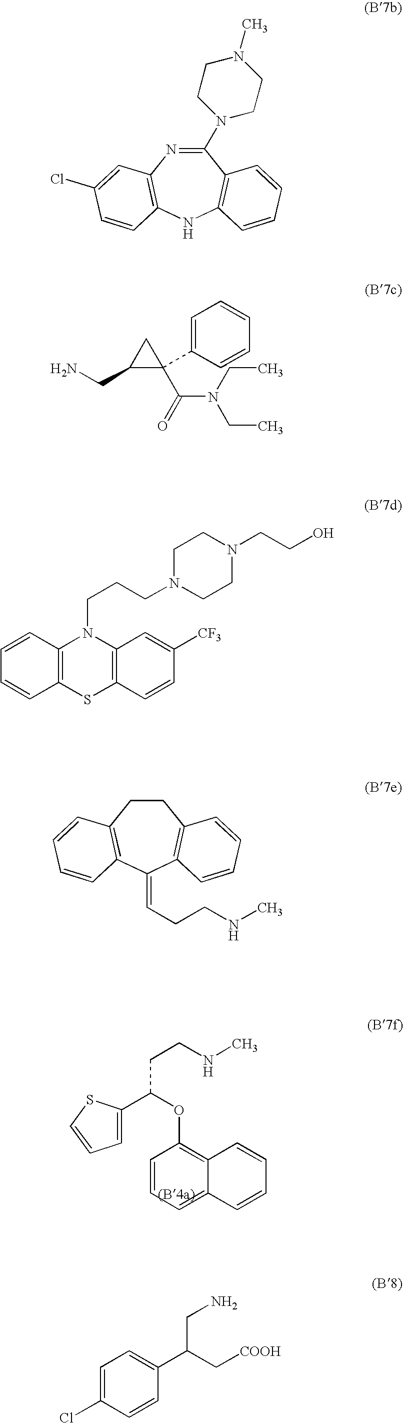 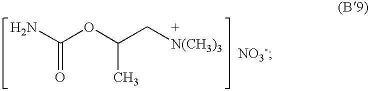
B′11) 3-(piperidin-1-yl)propyl 4 amino-5-chloro-2-methoxy benzoate (B′11a), 1-[4-amino-5-chloro-2-(3,5-dimethoxy phenyl)methyl oxy]-3-1-[2-methylsulphonylamino]ethyl piperidin-4-yl]-1-propanone (B′11b) 1-piperidinylethyl-1H-indol-3-carboxylate (B′11c), (S)-2-chloro-5-methoxy-4-(5-(2-piperidylmethyl)-1,2,4-oxadiazol-3-yl] aniline (B′11d).

8. Use according to claims 1-7, wherein the compounds inhibiting the phosphodiesterase C) salifiable with organic or inorganic acids are selected from the following: (C1) 1-[4-ethoxy-3-(6,7-dihydro-1-methyl-7-oxo-3-propyl-1H-pyrazol [4,3-d]-pyrimidin-5-yl)-phenyl]sulphoyl]-4-methyl-piperazine (Sildenafil), (C2) 2-(2-propyloxyphenyl)-8-azapurin-6-one (Zaprinast), (C3) 2,6-bis-(diethanolamino)-4,8-dipiperidino pyrimido [5,4-d]-pyrimidine (dipyridamol), (C4) 6-chloro-4-(1,3-dioxaindan-5-yl)methylamino-2 (4-carboxy-1-piperidinyl)-quinazoline, (C5) N-(phenylmethyl)-1-ethyl-1H-pyrazol-[3,4-b]-quinolin-4-amine, (C6) 1-(2-chlorobenzyl)-3-isobutyryl-2-propyl-6-aminocarbonyl-indol, (C7) l-benzyl-6-chloro-2-[1-[3-(imidazol-1-yl)propyl]indol-5-yl-amino carbonyl]benzimidazol, (C8) 2-(1-imidazolyl)-5-(phenyl)-4-(1,3-dioxaindan-5-yl) methyl aminopyrimidine, (C9) 6-ethynyl-4-(2-methoxyethyl)amino-2-(1-imidazolyl) quinazoline, (C10) 1-cyclopentyl-3-ethyl-6-(2-propoxy-phenyl)pyrazol[3,4-d]pyrimidin-4-one, (C11) 1-cyclopen-tyl-3-ethyl-6-(4-methoxybenzyl)-pyrazol-[3,4-d]-pyrimidin-4-one, (C12) 1,3-dimethyl-6-(2-propoxy-5-methansulphonamidophenyl)-1,5-dihydropyrazol[3,4-d]-pyrimidin-4-one, (C13) (6R,12aR)-2,3,6,7,12,12a-hexahydro-2-methyl-6-(1,3-dioxan-5-yl)pyrazino [2′,1′:6,1]pyri-do[3,4-b]indol-1,4-dione, (C14) 1-propyl-3-methyl-6-[2-propoxy-5-[(41-methyl-1-pyrazinyl) sulphonamido] phe-nyl]-1,5-dihydropyrazol [3,4-d]pyrimidin-4-one, (C15) 3-(4-amino carbonyl-1-piperidinyl)-6-cyan-8-(3-chloro-4-methoxyphthalazine, (C16) 2-(1-imidazolyl)-4-(1,3-dio-xaindan-5-yl) methylamino-7,8-dihydro-5H-thiopyran[3,2-d]pyrimidine (C17) 1-Cyclopentyl-3-ethyl-6-(3-ethoxypyrid-4-yl)-1H-pyrazolo(3,4-d] pyrimidin-4-one, (C18) 1-[3-(1-((4-Fluorophenyl)methyl]-7,8-dihydro-8-oxo-1H-imidazo[4,5-giquinazolin-6-yl]-4-propoxyphenyl] carboxamide.

9. Use according to claim 8, wherein the organic salts of C) are selected from oxalate, tartrate, maleate, succinate, citrate, glycinate, lysinate; and the inorganic anions are selected from nitrate, chloride, sulphate, phosphate.

10. Use according to claims 8-9, wherein the preferred anion is nitrate.

11. Use according to claims 1-10, obtained with formulations by oral, parenteral use containing one or more salts of the drugs of classes A)-C).

12. Nitrate salts of drugs compounds B′) of claims 1, 6 and 7, excluding the nitrate salts respectively of nifedipine, flunarizine, diltiazem.

13. Nitrate salts of drugs compounds C) of claims 1, 8 and 9 excluding sildenafil nitrate, zaprinast nitrate and dipyridamol nitrate.

14. Formulations according to claim 11.

15. Nitrate salts according to claims 12-13 for use as a medicament.
